(19) United States
(12) Patent Application Publication
(10) Pub. No.: US 2009/0149748 A1
(43) Pub. Date: Jun. 11, 2009

(54) Portable Pulmonary Injury diagnostic Devices And Methods

(75) Inventors: Martin Lenhardt, Hayes, VA (US); Kevin Ward, Glen Allen, VA (US)

Correspondence Address: WHITHAM, CURTIS & CHRISTOFFERSON & COOK, P.C., 11491 SUNSET HILLS ROAD, SUITE 340, RESTON, VA, 20190, US

(73) Assignee: VIRGINIA COMMONWEALTH UNIVERSITY, Richmond, VA (US)

(21) Appl. No.: 11/997,930

(22) PCT Filed: Aug. 25, 2006
(86) PCT No.: PCT/US2006/033368

§ 371 Date: Jul. 24, 2008

Related U.S. Application Data
(60) Provisional application No. 60/710,897, filed on Aug. 25, 2005.

(51) Int. Cl. A61B 5/08; A61B 8/00

(52) U.S. Cl. 600/437; 600/529

(57) ABSTRACT

Lung injury such as pneumothorax now can be diagnosed reliably, portably and quickly. Vibro-acoustic waves are sent through the chest and the resulting wave is measured. By analyzing attenuation characteristics determined by the geometry of the chest structures, a determination can be made of whether the patient's pleural space is healthy, contains air (pneumothorax) or contains fluid (hemothorax).

### FIELD OF THE INVENTION

The invention generally relates to emergency medicine, and especially relates to diagnosis of pulmonary injury including pneumothorax, hemothorax and other pulmonary injury.

### BACKGROUND OF THE INVENTION

Injuries to the thorax can lead to a number of injuries and death. These injuries include but are not limited to: chest wall contusions, rib fractures, pneumothorax, tension pneumothorax, hemothorax, hemopneumothorax, pulmonary contusions, cardiac contusion, penetrating injuries to the heart, and great vessels of the thorax and esophageal injury. These injuries may occur secondary to both blunt and penetrating mechanisms. Rapid death from these injuries usually takes the form of severe hypoxemia, hemorrhagic shock, or obstructive shock. The vast majority of chest injuries leading to these modes of death whether in the civilian trauma or combat setting involve pneumothorax (including tension pneumothorax), hemopneumothorax, and hemothorax which can be easily treated with needle or tube thoracostomy if correctly diagnosed (Shires 1994).

“Pneumothorax” refers to when air enters the pleural space between the lungs and the chest wall. In healthy individuals, this space is under negative pressure, so that the lungs can expand with the chest to breath. This pressure becomes positive when air enters the pleural space. This causes the lungs to collapse and can be life threatening. Treatment of pneumothorax is to insert a chest tube and extract the air inside the chest cavity.

When “hemothorax” occurs, fluid enters the pleural space, putting pressure on the lungs and making breathing more difficult. Hemothorax is as dangerous as pneumothorax, especially since the fluid that enters the pleural space is generally blood. When the blood volume in the pleural space exceeds 1500 ml, a “massive hemothorax” has occurred, which generally represents a major vascular injury. In treatment of hemothorax, a chest tube is inserted to drain the fluid. Additionally, there can exist combinations of pneumothorax and hemothorax termed hemopneumothorax which can be life-threatening; this condition usually occurs after trauma.

Treatment is relatively straightforward and can be performed in ambulances if needed. The dilemma with pneumothorax is that it sometimes is difficult to diagnose. In the field, paramedics tap on the chest, using their fingers, to listen for a hollow sound, which is not an accurate method and can be misinterpreted, especially in high ambient noise. In some cases, the chest tube has been inserted on the wrong side of the pneumothorax, which fails to cure pneumothorax and makes the problem even more life-threatening. The most accurate conventional method to determine if pneumothorax is present is to use either an x-ray or CT scan, which takes time, which tends to reduce patient survival.

Based on data from Vietnam, it is estimated that upwards of 20% of warfighters die of torso injuries that are potentially treatable. Approximately half of these involve tension pneumothoraces, with another unknown percentage involving injuries resulting in significant hemorrhage into the thorax (Bellamy 1984, Bellamy 1995).

Although body armor has certainly reduced the absolute degree of penetrating injuries to the chest, this will last only until new munitions are developed that overcome the ability of the armor to prevent penetration. In addition, new battlefield threats such as those involving improvised explosive devices have may be resulting in an increase in behind body armor trauma via blunt force mechanisms. Sufficient blunt force may result in pneumothorax, tension pneumothorax, hemopneumothorax and hemothorax by a number of mechanisms, including rib fractures and intercostals and lung vessel injury (Ling 1999).

Although treatment of pneumothoraces ranging from tension to hemothorax is straightforward and a skill readily teachable to advanced field medics, the diagnosis of the injury based on physical exam is not. Except for visualization of gross violation of the chest cavity, palpation of chest wall crepitance (from pleural air entering the chest subcutaneous tissue), palpation of flail chest wall segments, or visualization of tracheal deviation, the physical exam is extraordinarily limited in its ability to diagnose most pneumothoraces. Auscultation of the chest to identify absent or diminished breath sounds and/or percussion of the chest to detect differences in conduction of mechanical energy are notoriously inaccurate even at sophisticated civilian trauma centers. Even in the case of penetrating trauma, auscultation has only a 58% sensitivity and is capable of missing hemothoraces up to 600 cc and hemopneumothorax up to 800 cc (Chen 1997). In the setting of combat injury, especially during the first medic-wounded warfighter encounter, the sensitivity of auscultation will be reduced even further due to noise, need for rapid movement, overlying garments, and mental stress.

Although not as well studied, traditional percussion in this environment is unlikely to greatly improve the potential to make a correct diagnosis (Winter 1999). Again, environmental issues (such as noise, access to the entire chest wall, etc.) will likely prevent this technique from being very useful. Also, it is not uncommon for the injured to have bilateral pneumothoraces including hemothorax and hemopneumothorax, making the use of an uninjured lung and chest wall for comparison to the injured side not possible.

For these reasons, plain film chest radiography has served as the historical gold standard to diagnose pneumothoraces of various types. This technique is noninvasive and provides rich information, but is not practical as a combat medic or a civilian medic tool. Conventional attempts to create a portable bedside diagnostic solution have used high frequency ultrasound. Even though high frequency ultrasound (MHz range) is portable and capable of detecting pneumothoraces, it is a highly operator dependent technique requiring significantly clinical training and expertise to perform (Chung, 2005, Jaffer 2005) Furthermore, high frequency ultrasound seems not to be meeting portability and ruggedness requirements needed for a field device nor to be satisfying cost concerns, so that a high-frequency ultrasound has not been viewed as a practical option for routine combat medic deployment.

Liberal performance of needle or tube thoracostomy when in doubt of the diagnosis, although occasionally life saving, and many times harmless, has its dangers including actually creating a pneumothorax (including tension pneumothorax), causing significant hemorrhage (if intercostal or lung vessels are injured), lung damage, and chest and systemic infection. Thus, ideally, treatment of pneumothoraces would take place only when the correct diagnosis made, or when the benefit of “suspected” treatment significantly outweighs the risk such as might occur if air-transport is required (Haid 1992).

Because pneumothoraces of various types are the main treatable life-threatening chest injury to the warfighter, development of a portable, small, lightweight and easy to use diagnostic device available for use by the combat medic would be very much wanted in order to enhance the medic's ability to save lives at the time of injury. Such a device also would be wanted for other settings, such as mass casualty triage and evacuation scenarios. Because supplies are limited in the multiple casualty encounter, better portable diagnostics will mean improved allocation of resources. However, a portable device (such as a hand held device) which can function in high ambient noise, has proven to be a challenge to design and construct and before this invention apparently has not been provided. A significant problem has been that typically large transducers and related amplifiers etc. are needed to propagate frequencies in the infrasound range (<20 Hz) needed to drive internal organs into resonance.

Considered more generally, whether caused by trauma, internal diseases, or spontaneously, pneumothorax and hemothorax are potentially life threatening illnesses which conventionally primarily diagnosed with x-rays, CT scans, and ultrasound imaging (when available). While these methods are generally reliable, they are not always available to an injured patient. Life threatening pneumothoraces, such as tension pneumothorax, must be treated quickly. When diagnosed correctly, pneumothorax and hemothorax can be quickly mended through insertion of chest tubes. Conventional X-rays, CT scans, and ultrasound imaging require large scale equipment and are not always dependable. Every year, 6800 people in the United States have a spontaneous pneumothorax (Healthgrades). When this occurs, the patient cannot breath sufficiently and can have lowered cardiac output. If certain pneumothoraces are not diagnosed and treated, they may result in death. Between 2000-2002, 2,347 patients died from iatrogenic pneumothorax (Heathgrades). Many of these deaths could have been prevented if diagnosing pneumothorax were more convenient. Conventionally, x-rays or CT scans are used to identify pneumothorax, which requires large scale equipment and can be time consuming.

A more reliable, portable, and faster result producing method to diagnose pneumothorax is much wanted.

However, conventional thinking before this invention, relying on certain specific resonant frequencies from a diagnostic perspective, imposes limitations.

The following literature is generally mentioned as background:

U.S. Pat. No. 4,122,842 issued Oct. 31, 1978 to Pikul for “Pulmonary diagnostic instrument including breath transducer.”

U.S. Pat. No. 4,796,639 issued Jan. 10, 1989 to Snow et al. for “Pulmonary diagnostic system.”

U.S. Pat. No. 6,261,238 issued Jul. 17, 2001 to Gavriely for “Phonopneumograph System.”

U.S. Pat. No. 6,368,286 issued Apr. 9, 2002 to Whitman et al. for “Pneumothorax detector.”

U.S. Pat. No. 6,436,057 issued Aug. 20, 2002 to Goldsmith et al. for “Method and apparatus for cough sough analysis.”

U.S. Pat. No. 6,533,730 issued Mar. 18, 2003 to Strom for “Method for assessing pulmonary stress and breathing apparatus employing the method.”

U.S. Pat. No. 6,595,923 issued Jul. 22, 2003 to Mansy et al. for “Method and apparatus for detection of air cavities in a body.”

U.S. Pat. No. 6,650,924 issued Nov. 18, 2003 to Kuth et al. for “Method and apparatus for the automatic recognition of pneumothorax.”

U.S. Pat. No. 6,718,975 issued Apr. 13, 2004 to Blomberg for “Method of assessing pulmonary stress and breathing apparatus.”

### REFERENCES

Bellamy R F: The causes of death in conventional land warfare: implications for combat casualty care research. Mil Med; 1984; 149:55-62.
Bellamy R F: Combat trauma overview, Anesthesia and perioperative care of the combat casualty. Ed: Zajtchuk R, Grnade C M. Washington D.C. Office of the Surgeon General, 1995, pp 1-53.
Chan S S. Emergency bedside ultrasound to detect pneumothorax. Acad Emerg Med 2003; 10:91-94.
Chen S C et al: Hemopneumothorax missed by auscultation in penetrating chest injury. J Trauma. 1997; 42:86-89.
Chung M J, et al. Value of high-resolution ultrasound in detecting a pneumothorax. Eur Radiol 2005; 5:930-935.
Gaillard M, Herve C, Mandin L, Raynaud P: Mortality prognostic factors in chest injury. J Trauma. 1990; 30:93-96.
Haid M M, et al: Air transport and the fate of pneumothorax in pleural adhesions. Thorax 1992; 47:833-834.
Jaffer U, McAuley D. Best evidence topic report. Transthoracic ultrasonography to diagnose pneumothorax in trauma. Emerg Med J. 2005; 22:504-505.
Legome E, Pancu, D. Future applications of emergency ultrasound. Emerg Med Clin N Am 2004; 22:817-827.
Ling G, Day B, Rhee P, Ecklund J: In search of technological solutions to battlefield management of combat casualties. SPIE 1999; 3712:1-8.
Ling G. et al,. Diagnosis of pseumothorax using a microwave based detector. SPIE 2001; 4368: 146-151
Ma O J, Mateer J R. Trauma ultrasound examination versus chest radiography in the detection of hemothorax. Ann Emerg Med 1997; 29:312-316.
Parsons C J, Bobechko W P: Aeromedical transport; its hidden problems. Can Med Assoc J. 1982; 126:237-243.
Shires G T, et al: “Trauma” in Principles of Surgery, S. I. Schwartz, G. T. Shires, and F. C. Spencer, Editors. Pp. 175-224, McGraw-Hill, New York, 1994.
Soldati G. Iacconi P. The validity of the use of ultrasonography in the diagnosis of spontaneous and traumatic pneumothorax. J Trauma 2001; 51(2):423.
Winter R, Smethurst D. Percussion, a new way to diagnose pneumothorax. Br J Anaesth. 1999; 83:960-961

### SUMMARY OF THE INVENTION

Lungs exhibit acoustic resonance, that is, they vibrate best on a small range of frequencies based on their mass and geometries. Lungs also pass (transmit) vibratory frequencies based on the same characteristics that permit them to exhibit acoustic resonance as well as based on their coupling to the surrounding tissue. The present invention, instead of only being interested in certain specific resonant frequencies as in conventional pulmonary diagnostic technologies, exploits properties and characteristics of a range of frequencies passing through the structure, and applies differences under different disease or injury conditions for diagnostic uses. This innovative use of a range of frequencies, rather than a specific frequency, has lead to inventive devices, systems, diagnostic methods, tissue analysis methods, and handheld applications.

For example, pneumothorax now can be diagnosed reliably, portably and quickly, by sending vibro-acoustic waves through the chest and measuring the resulting wave(s). By analyzing attenuation characteristics determined by the geometry of the chest structures, a determination can be made of whether the patient's pleural space is healthy, contains air (pneumothorax) or contains fluid (hemothorax).

The present inventors have invented, inter alia, a handheld device based on the frequency range 20 kHz to 50 kHz which may be used, e.g., by corpsmen to diagnose lung injury (such as, e.g., pneumothoraces) at the point of wounding in conscious and unconscious combat casualties. An exemplary inventive device uses low frequency ultrasound or sound waves, is automated, and includes an easy-to-use interface and readout, making the device non-operator dependent and requiring minimal training and experience. Early use of such an inventive device on an injured patient may improve chances of survival, such as, e.g., in the combat setting. The inventive technology also can be used in civilian trauma care including use by paramedics in the field and emergency physicians and surgeons in the trauma centers and community emergency departments.

The invention in one preferred embodiment provides a method for diagnosing pulmonary injury, comprising: interrogating at least one pulmonary tissue which is within a patient with at least one wave having a frequency in a range of about 20 kHz to 50 kHz (such as, e.g., a wave having a frequency above 30 kHz, etc.), wherein the at least one wave is selected from the group consisting of: a continuous wave, a pulsed wave, an amplitude modulated (AM) wave, and ultrasonic noise; automatically detecting results of the interrogating step; from which detected results a diagnosis may be made of whether a pulmonary injury (e.g., pneumothorax, hemothorax, hemopneumothorax, pulmonary effusion, pulmonary contusion, etc.) is present in the patient; such as, e.g., methods wherein the interrogating comprises interrogating with a continuous wave; methods wherein the interrogating comprises interrogating with a pulsed wave; methods wherein the interrogating comprises interrogating with an AM wave; methods wherein the interrogating comprises interrogating with an ultrasonic noise; methods wherein the tissue interrogating is noninvasive; methods including placing at least one probe on a region of soft tissue of a patient, methods including diagnosing pneumothorax; methods diagnosing hemothorax or hemopneumothorax; methods including a percussion step; methods practiced using a handheld device and wherein pneumothorax, hemothorax or hemopneumothorax is diagnosed; methods in which pneumothorax, hemothorax or hemopneumothorax may be diagnosed in an environment with high ambient airborne noise; methods including a step of diagnosing whether pulmonary injury is present or absent in the patient; methods including an automated diagnostic step; methods including an FFT step and/or spectral analysis step; etc.

In another embodiment, the invention provides a portable medical device for diagnosis of lung injury, comprising: a wave and/or noise generator generating at least one wave having a frequency in a range of about 20 kHz to 50 kHz, wherein the at least one wave is selected from the group consisting of: a continuous wave, a pulsed wave, an amplitude modulated (AM) wave, and ultrasonic noise; an interface through which the low frequency wave is delivered to at least one lung, and a detection component which detects a signal that results from applying the low frequency wave to the lung; such as, e.g., portable medical devices wherein components inconsistent with the device having a portable size are excluded; portable medical devices that are handheld size; portable medical devices that are waterproof; etc.

In another preferred embodiment, the invention provides a portable medical device for diagnosis of lung injury, comprising: a wave and/or noise generator generating at least two different frequencies simultaneously (such as, e.g., each frequency being in a range of about 20 kHz to 50 kHz), an interface through which the at least two different frequencies simultaneously are delivered to at least one lung, and a detection component which detects a signal that results from applying the at least two frequencies to the lung; wherein components inconsistent with the device having a portable size are excluded. Examples of a frequency source for each different frequency used in an inventive medical device is, e.g., a continuous wave, a pulsed wave, an amplitude modulated (AM) wave, ultrasonic noise, etc. Frequency sources used in an inventive medical device may be the same. A mixture of frequency sources may be used in an inventive medical device.

In a further preferred embodiment, the invention provides a method of diagnosing lung injury, comprising: generating at least two different wave and/or noise frequencies simultaneously, through an interface simultaneously delivering the at least two different frequencies to at least one lung, and detecting a signal that results from the at least two frequencies being delivered to the lung.

### BRIEF DESCRIPTION OF THE DRAWINGS

The foregoing and other objects, aspects and advantages will be better understood from the following detailed description of the preferred embodiments of the invention with reference to the drawings, in which:

FIG. 1 includes spectral amplitudes. The left panel is an example of an amplitude modulated (AM) pulse in water (like skin, left panel) and water and air (like pneumothorax). Note the attenuation of the recorded signal (blue—middle data) and the change in the spectral amplitude (red—bottom data). The emitter intensity remains constant (yellow—top data). Ceres Biotechnology equipment was used. The top (yellow in the original) tracing is the emitter signal and the middle (blue in the original) is the receiver signal. The FFT is on the bottom (in red on the original).

FIG. 2 shows connection of an inventive device as in Example 1 herein.

FIG. 3 shows a spectrum of continuous wave (CW) noise through the chest of one pig. Note the clear separation of tracing for the higher, ultrasonic, frequencies (large circle on right side). In the large circle, the three curves which separate out on the top around y=−60 dB (re: 1 Volt) correspond to healthy results, as do the separated-out curves which approach y=−100 dB.

FIG. 4 shows acoustic attenuation of averaged tracings for hemothorax (top in FIG. 3), pneumothorax (middle in FIG. 3) and health controls (bottom in FIG. 3).

FIG. 5 shows Low frequency response of the recorded sound through pig tissue using the MSI transducer system, individual trials.

FIG. 6 shows a summary of the averaging advantage in signal resolution.

FIG. 7 shows simulation of the proposed device in the Emergency Room.

FIG. 8 are waveforms used in an exemplary embodiment of inventive acoustic percussion for a portable pulmonary injury diagnostic device.

FIG. 9 shows receiving and emitting transducers connected to an FFT, as discussed in inventive Example 2.

FIG. 10 is a graph showing comparison between acoustic properties of water and air balloons. (See Ex. 2A) FIG. 10A is derived from FIG. 10 and is averaged.

FIG. 11 is a graph showing comparison between healthy human and pig acoustic responses. FIG. 11 is derived from FIG. 11 and is average. Note: the spike at 2.2 kHz was the result of external device noise.

FIG. 12 is a graph of dB/cm attenuation measurements (Example 2B).

FIG. 13 is a graph of initial signals produced, before damping, when the two transducers are connected to each other. (Example 2B)

FIG. 14 is a graph showing a comparison of four different setups (initial signal; hemothorax; pneumothorax; healthy). (Example 2B) Note: the spike at 2.2 kHz was the result of external device noise.

FIG. 15 is a graph showing the average of each pleural space test. (Ex. 2B)

FIG. 16 is a graph showing frequency results from 500-2000 Hz (Ex. 2B).

FIG. 17 is a graph showing attenuation across the skin compared to the emitter transducer coupled to the receiver transducer. (Example 2B)

FIG. 18 is a graph showing transmission loss due to impedance of collapsed and inflated lungs. (Example 2B)

FIG. 19 is a graph showing chest wall transmission loss due to impedance, compared to the initial signal and the healthy results. (Example 2B)

FIG. 20 is a table of the speed of sound and half value layer characteristics of different mediums. (Webster.)

FIG. 21 is graphical comparison of measurements (Example 2B) for initial signal; chest wall; hemothorax; collapsed lung; inflated lung; pneumothorax; and healthy.

FIG. 22 is a statistical analysis table (Example 2B).

FIG. 23 is a histogram comparison.

FIG. 24 is a probability plot comparison.

FIG. 25 is a stem leaf data chart.

FIG. 26 includes equations a), b), c).

FIG. 27 is an inverse fast Fourier transform. FIG. 27A is based on FIG. 27 and is scaled. FIG. 27B is a table of statistical data.

FIG. 28 is a bar graph showing right and left thorax measurement comparison (˜80 dB scaled).

FIG. 29 is a diagram of a pneumothorax detecting device in an exemplary embodiment of the invention.

FIG. 30 includes a cross-sectional diagram (left) of an exemplary waterproof structure of piezoelectric ceramic rods embedded in a polymer matrix that may be used in the invention, and a photograph (right) of a transducer for which the diagram corresponds.

FIG. 31 is a photograph of a portable non-invasive pneumothorax device according to an exemplary embodiment of the invention.

### DETAILED DESCRIPTION OF THE PREFERRED EMBODIMENTS OF THE INVENTION

Lungs exhibit acoustic resonances, that is, they vibrate best on a small range of frequencies based on their mass and geometries. Lungs also pass (transmit) vibratory frequencies based on the same characteristics that permit them to exhibit acoustic resonance as well as based on their coupling to the surrounding tissue. Conventional thinking before this invention was to rely only on certain specific resonant frequencies from a diagnostic perspective. However, the present invention instead exploits properties and characteristics of a range of frequencies passing through the structure, and applies differences under different disease or injury conditions for diagnostic uses.

“Low frequency” mentioned herein refers to frequencies under 50 kHz, i.e., “low frequency ultrasound” means ultrasound using waves less than 50 kHz. A preferred range of low frequency waves is a range of between about 20 kHz and 50 kHz, most preferably a range of low ultrasonic frequencies between 30 kHz and 50 kHz. The frequency range in a particular application is not limited to the ranges stated herein, and a frequency range is to be chosen depending on geometries of an individual patient. A preferred example is as follows. Typically initial use is of noise, which contains a wide range of frequencies. Those frequencies that are amplified due to individual specific resonances can be identified. Once so identified, fine scale analysis using frequencies within the range of resonance are applied for greater degree analysis.

In the invention, a range of frequencies (i.e., at least two frequencies) is simultaneously applied to a patient to be diagnosed. The different frequencies in the range may belong to waves which are the same type or a mixture of two or more different types.

Examples of types of waves (i.e., frequency sources) which may be used in practicing the invention are, e.g., continuous waves; pulsed (pulsing) waves (such as, e.g., mechanical superimposed pulses, sound superimposed pulses, non-superimposed pulses, etc.); amplitude modulated waves; ultrasonic noise (also called “noise bands”); etc.

An example of using continuous waves in an inventive diagnostic method is using at least two continuous waves of different frequencies, where the first frequency and the second frequency are in the range of 20 kHz to 50 kHz, and the at least two waves are applied simultaneously to the patient to be diagnosed.

Alternately, instead of or in addition to using continuous waves, pulsed waves may be used, i.e. at least two pulsed waves of different frequencies in the range of 20 kHz to 50 kHz. For example, a mixture of continuous wave(s) and pulsed wave(s) may be simultaneously applied to the patient to be diagnosed.

Superimposition of a pulse over at least one continuous wave is referred to herein as “percussion.” Use of pulsing advantageously introduces a frequency splash (i.e., a spreading of frequencies due to the onset and off set of the pulsed signal) and that extra spectra can be very helpful in establishing non-fluid interfaces such as air. Pulses may be used in a form that is superimposed (such as, e.g., mechanical superimposed pulses, sound superimposed pulses) on another wave form, or may be non-superimposed.

An example of using amplitude modulated (AM) waves in an inventive diagnostic method is using at least two amplitude-modulated waves of different frequencies in the range of 20 kHz to 50 kHz, and the at least two waves are applied simultaneously to the patient. The use of amplitude modulation and inducing parametric demodulation advantageously allows for the use of inexpensive transducers to stimulate at lung resonance. AM waves are discussed further below, see Example 1.

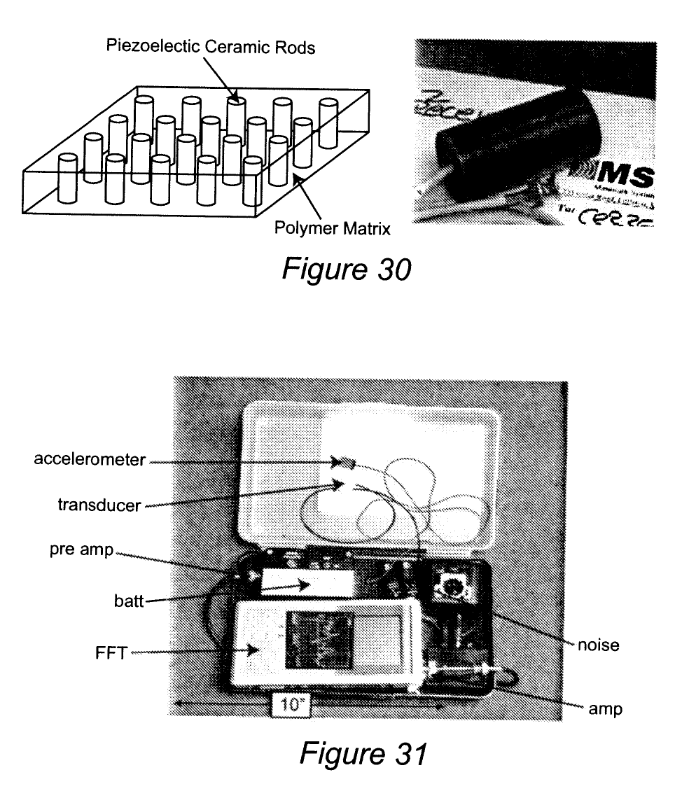

“Ultrasonic noise” referred to herein means white noise with a passband of ˜20-100 kHz. An example of using ultrasonic noise in an inventive diagnostic method is using ultrasonic noise comprising at least two different frequencies in the range of 20 kHz to 50 kHz. Use of ultrasonic noise is preferred for providing a fast method of finding an optimal frequency to diagnose pneumothorax or hemothorax. A noise band (e.g., ultrasonic noise with a 20-50 kHz spectrum) may be generated with a specialized transducer, such as, e.g., a multi-rod piezoelectric transducer embedded in a polymer case (see FIG. 30). The transducer should be waterproof, a necessary safety feature for use in a trauma application.

Whatever the type of wave (i.e., continuous, pulsed, amplitude modulated, ultrasonic noise), it is preferred to use a frequency range comprising more than just than two different frequencies, such as, e.g., at least three different frequencies, at least four different frequencies, etc. Each type of wave can be used in several combinations to help make the diagnosis. The choice depends upon how each interacts with the tissue to resolve the question. Continuous noise then pulse noise is a typical approach.

Application to a patient of a range of different-frequencies according to the invention may be accomplished in different ways, most preferably non-invasively. In the inventive methods, systems and devices, contact (vibration) ultrasound, not airborne ultrasound, is used because of the following considerations. The ratio of the characteristic impedances (Zo) of any two media on either side of an interface determines the degree of reflection and refraction or transmission of the incident wave. The characteristic acoustic impedance of a medium is the product of the density (rho) and the speed (c) of sound in that medium. The extent to which ultrasonic energy is transmitted or reflected at an interface separating two continuous media is determined by the ratio of the characteristic acoustic impedances. The closer this impedance ratio is to 1, i.e. impedances matched, the more energy is transmitted into the second medium and the less is reflected from the interface. At an interface between human tissue and air, only ˜0.01% of the incident energy is transmitted, the remainder being reflected. For this reason, contact (vibration) ultrasound is used in the invention.

For example, the different-frequencies of waves may be applied to a patient via at least one transducer in contact with the patient's skin, such as on the patient's chest. In a preferred embodiment, waves are sent up a patient's chest by placing a transducer under the patient's ribs by the diaphragm and recording with the transducer over soft tissue near the clavicle.

In another embodiment, the waves used may be caused to travel down the respiratory tree through the patient's trachea, e.g., the different frequencies may enter tracheally (and, after the different frequencies pass through the lungs, the response is measured on the chest wall). Alternately, the wave-form may be applied non-tracheally and then after having passed through the lungs may exit tracheally. Many patients needing to be diagnosed for possible pulmonary injury already will have a breathing tube in place, and the practice of the invention preferably takes into account the tracheal tube (e.g., different-frequencies of this invention can be applied via the tracheal tube and via the trachea, and the returned wave form can exit via the chest; alternately, different-frequencies can be applied via the chest wall and sent up the trachea). A vibrational source on the trachea can be compared with surface transducers on the patient's chest at various locations. Alternately, a patient's mouth (oropharynx) may be used for delivering and receiving signals (e.g., propagating a signal from that point down the trachea, etc.), such as, e.g., without an endotracheal tube or nasogastric tube being in place, etc. A sensor array may be used for receiving signals.

Application of the range of frequencies to the patient's pulmonary tissue results in a resulting signal. For example, when ultrasound is transmitted through a heterogeneous medium such as soft tissue, its intensity is reduced or attenuated through a number of mechanisms, including scattering, absorption, reflection, diffraction, and refraction. Absorption of ultrasound occurs when the ordered vibrational energy of the energy is dissipated into heat. In the invention, ultrasound attenuation is measured. By measuring attenuation, pneumothorax and other non-healthy tissue may be differentiated.

Advantageously, in the invention, airborne ambient noise does not interfere with hearing or otherwise detecting and processing the resulting signal because transducers are coupled to the patient's body, such that only bodily ambient noise, not airborne ambient noise, is relevant to signal processing. In some embodiments, a resulting signal can be directly listened to (e.g., when ultrasound frequencies are used that are loud enough to be heard and are presented as vibration to be listened to). Preferably, the resulting signal is automatically processed into a visual display which is viewable by a user.

Examples of an interface useable in the inventive medical devices are, e.g., an interface via which the wave is delivered non-invasively to a patient; an interface via which the wave is delivered via a region selected from the group consisting of: a trachea of a patient and a soft tissue region of a patient; an interface via which the low frequency wave is delivered via a trachea of the patient and detected on the thoracoabdominal or is received at the trachea after having been delivered to the patient's thoaracoabdominal surface; an interface via which the low frequency wave is delivered or received from an endotracheal tube which is in the patient's trachea and respectively received or delivered from the patient's thoracoabdominal surface; an interface via which the low frequency wave is delivered or received from a nasogastric tube which is in the patient's esophagus and stomach and respectively received or delivered from the patient's thoracoabdominal surface; an interface via which the low frequency wave is delivered or received from a mouth (oropharynx) of a patient; etc.

Examples of a wave and/or noise generator useable in the inventive medical devices are, e.g., a wave and/or noise generator that comprises a simulator system simulating at least one continuous wave; a wave and/or noise generator that comprises a simulator system simulating at least one pulsed wave; a wave and/or noise generator that comprises a simulator system simulating at least one demodulated wave; a wave and/or noise generator that comprises a simulator system simulating at least one amplitude modulated (AM) wave; a wave and/or noise generator that comprises a non parametric propagator; a wave and/or noise generator that comprises a simulator system simulating ultrasonic noise; etc.

Examples of pulmonary injury which may be diagnosed using the inventive methods, devices and systems are, e.g., pneumothorax, hemothorax (i.e., collection of blood), hemopneumothorax (collection of blood and air), pulmonary effusion (i.e., collection of fluid other than blood in the chest), pulmonary contusion (i.e., injury to the lung tissue itself), etc.

In a particularly preferred embodiment, the invention may be practiced in portable handheld diagnostic devices. “Portable, handheld” herein refers to a size of ˜5 inches by 4 inches by 2 inches or smaller, and a weight of ˜3 pounds or less.

### EXAMPLE 1

This Example uses a demodulating technique based on propagating an amplitude modulated (AM) signal in a non linear system as human soft tissue. Non parametric demodulation will result in the low frequency modulator and the carrier in the tissue. In this Example, a small transducer may be easily incorporated into a hand held architecture. The carrier in this Example is low frequency ultrasonic tones or low frequency ultrasonic noise. Frequencies between 25 and 50 kHz differentiate various induced fluid conditions in pig lung. The low frequency ultrasonic tones or low frequency ultrasonic noise (i.e., the stimulation) may be applied anywhere over the thorax. Preferably, the tracheal area is stimulated. Lung sounds may be recorded.

### Low Frequency (>20 kHz) Ultrasound

Acoustic energy may be transformed into several other forms of energy, which may exist at the same time as heat, sonic and ultrasonic frequencies. When ultrasound is absorbed by tissue it produces alternate areas of compression and rarefaction in the tissue and the spectral changes (frequency specific amplitude) can reflect the biological condition of the tissue. The intensity of the ultrasound is reference to power/area, however we measure the intensity in acceleration (1 m/s²).

Ultrasonic mechanical vibrations occur above the upper frequency limit of human audibility (>20 kHz). Ultrasound consists of a propagating wave in the tissue with direct contact vibration. Direct contact vibration is important in that only the ambient noise in the tissue is of concern. However, the human body does absorb sound once its impedance mismatch has been exceeded, usually about 50-60 dB depending upon the frequency. Thus, an inventive device can function in battle field noise. Ultrasound may propagate in different modes but in tissue, compressional (longitudinal) waves dominate. If however, a longitudinal wave propagates in soft tissue (such as lungs), transverse waves can be launched in the bone or ribs in the inventive methods, systems and devices. A wave propagating in a medium is characterized by acoustic energy (acceleration), as well as particle displacement, particle velocity, and particle acceleration. Only acceleration is a concern in the preliminary measurement. Ultrasonic vibration amplitude, as measure in acceleration (1 m/s²) will decay in reference to the source level and the characteristics of the medium (tissue). In this example only acceleration will be used to measure the ultrasound and sonic energy. While technically this example uses ultrasound it is well below the imaging frequencies and falls into the classification of contract ultrasound as defined by OSHA. Further 1 W/s² is equal to 160 dB SPL, far above the energy used to detect a pneumothorax. Acceleration is not the speed of vibration but is the speed with which the wave propagates through the medium; and is a constant that depends on the physical properties of the medium. Although previous investigators have tried to correlate changes in speed with injury, the equipment to measure precisely arriving waves and their multiple reflections has been prohibitive and their attempts did not result in a hand held device for diagnosing lung injury.

### Continuous (CW) and Amplitude Modulated (AM) Waves

In a basic embodiment, continuous wave (CW) noise was used to probe the chest. A continuous wave is passed though the chest, optimized for the lung and the attenuation of the wave is measure, which reflects the biological condition of the tissue.

Preferably, an alternative innovation is to use amplitude modulation (AM), i.e., a CW frequency is used as a carrier and is multiplied by a lower frequency wave.

### Non Parametric Demodulation of Low Frequencies

If a low frequency wave is multiplied by a high frequency carrier and the AM signal is passed thorough a non linear medium as a the body the low frequency, the modulator will demodulate resulting in both the low frequency sound and the carrier present in the tissue. If for example a tone of 20 Hz, near the resonant characteristic of the chest is modulated on an ultrasonic carrier the non linear characteristics of the lung will demodulate the AM signal such that the low frequency sound can now interact with the lung near its natural resonance. This approach, relying on non parametric propagation will allow the use of frequencies near the organ's resonance without the need for large transducers, prohibitive in battlefield applications. In the example below 8 Hz is multiplied by 45 Hz. Eight Hz will demodulate due to the non linearity of the propagating medium, in our case skin; resulting in two frequencies: 8 and 45 Hz. (Westervelt P J, The theory of steady forces caused by sound waves, Journal of the Acoustical Soc. of Am., 23, 4, 312-314 (1951); Westervelt, P J, Parametric acoustic array, Journal of the Acoustical Soc. of Am., 35, 4, 532-537 (1963).

TABLE 1
Amplitude Modulation (AM)

Modulation = 8 by 45 Hz
AM = (1 = cos Wt) × cos wt
W = 2 pF [modulating~] and w = pf [carrier~]
Demodulation = 8, 45 Hz
Demodulation W = W^{a} FW [f2 (t)]
^{a}is an exponent = 1.85

### Signal Specification

Low frequency ultrasound is propagated from the transducer, the beam size remains relatively constant (the near field), or Fresnel zone which extends from the transducer to a distance equal to D²/4 lambda (when D is much greater than lambda); an intensity analysis of the near field of a vibrating transducer will be made for this application by using accelerator arrays. A potential problem in specifying intensity are possible standing waves induced between the ribs and related tissue.

Standing waves can occur when CW ultrasound is propagating into a confined space, such that the waves are reflected back from an interface and travel in opposite directions. Standing waves can be induced at the bone/tissue or tissue/gas interface, both present in the chest. The results are minima and maxima of the wave amplitude, called “nodes” and “antinodes” which can be specified using accelerometers arrays over the chest.

The following basic signal types were evaluated:
Pure tones [continuous waves (CW)]
Tonal pulses
Noise [continuous wave (CW)]
Pulsed noise
Amplitude modulated (AM) noise
AM pulsed noise (AM noise, pulses)

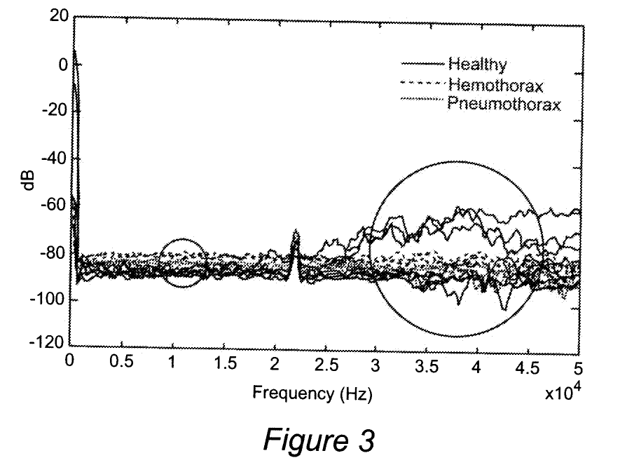

An example of an AM noise (30 kHz carrier modulated by noise 3-10 kHz) pulse in a tank is depicted in FIG. 3. Baseline is on the left and air space, simulating a pneumothorax, is on the right. The pneumothorax pattern is clearly discernable from baseline.

### EXAMPLE 1A

Assessment of Propagation Patterns: Fourier analysis was used to determine the spectra of signals with various transducer placements in a water/air simulated environment using a waveguide or tank.

Two female adult Hampshire pigs weighing about 50 kg were used in a pilot study of a lab version of the novel hand held battle field interthoracic device. The animal study was approved by the local vertebrate animal review board. Each pig was sedated with ketimine and pentobarbital. The stimulating sensor 20 and recording sensor 22 are depicted in FIG. 2. Vibratory probes (transmitter and receiver pair) were positioned around the chest to measure the acoustic properties of each normal chest/lung. Care was taken to avoid placing the ribs in the acoustic beam. Data was acquired using a real time spectral analyzer in the surgical suite and processed off line via MatLab program developed for quantification. Immediate spectra plots were available in the lab. Two other experimental conditions were induced, hemothorax and pneumothorax. The hemothorax was created by filling the lung with saline and the pneumothorax was created by administrating air via pleural puncture.

The signals were continuous (CW) wide band noise (0.6-50 kHz) generated by the spectrum analyzer. The receiver probe fed the recorded signal back into the analyzer. The signal amplitude was sampled on the input and output of the cheat wall (mass loaded). A calibrating signal was also sent thought the chest.

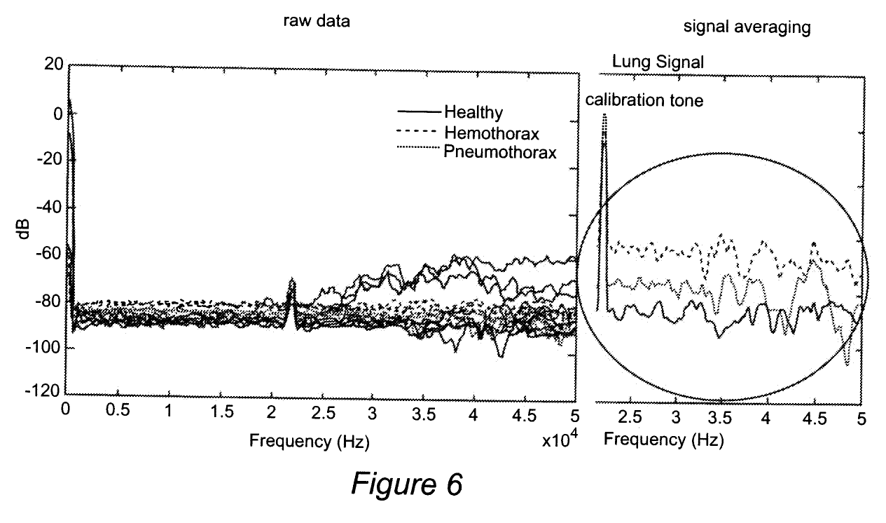

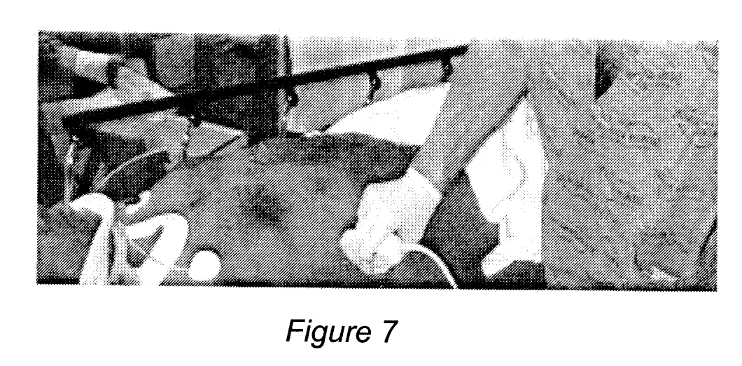

The raw data, unprocessed other than filtering due to the mechanical properties of the emitter/receiver pair is presented in FIG. 6.

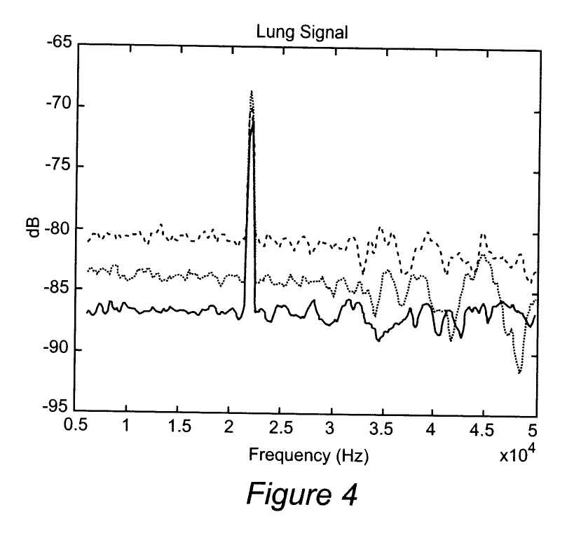

Referring to FIG. 3, note the clear separation of tracing for the higher, ultrasonic, frequencies (large circle on right side). In the large circle, the three curves which separate out on the top around y=−60 dB (re: 1V) correspond to healthy results, as do the separated-out curves which approach y=−100 dB. Still referring to FIG. 3, the frequencies passed through the lung in the normal (healthy), hemothorax and pneumothorax conditions are all broadband as expected. Note while there is separation of the spectra for the data runs on each animal for all three conditions, there is better resolution of the difference above 30 kHz, especially between the healthy and pathological conditions. While the separation between healthy and pathological conditions exists on most trials, there was some occasional overlap. To obtain better resolution the individual trials were averaged and the result is depicted in FIG. 4. Referring to FIG. 4, all average tracings (7-8 trials/condition) are clearly different although the range is only 5.5 dB (re: 1 volt)[see Table 2].

TABLE 2
Attenuation data

| Condition | Attenuation in dB (re: 1 V rms) |
|---|---|
| Emitter source | −69.41 |
| Health control | −86.52 |
| Pneumothorax | −84.24 |
| Hemothorax | −81.00 |

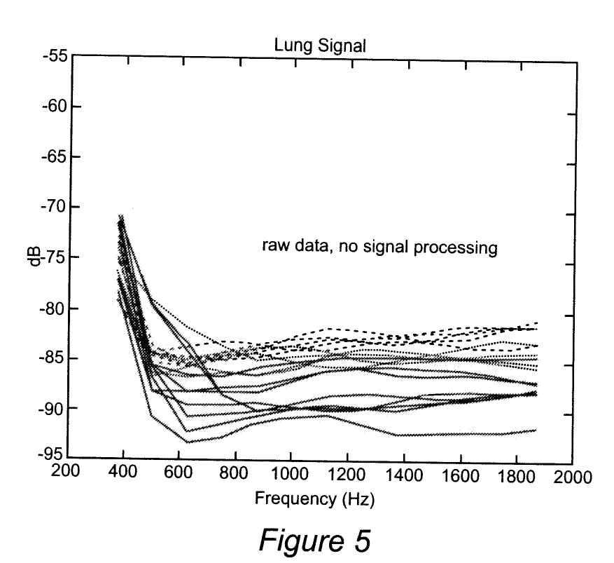

Resolution of low sonic frequencies in lung for diagnosis is also possible. It is clear from FIG. 4 there is considerable overlap in the tracings below 30 kHz (see FIG. 5).

Repeating averaging improved the accuracy of the ultrasonic frequencies in detecting a pneumothorax in a pig model; however averaging parsed the waves for the lower sonic frequencies as well. These data are summarized in FIG. 6.

Thus the combination of low frequency sonic vibration and ultrasonic vibration are helpful in detecting a pneumothorax in a pig preparation in just a few seconds.

The device of this Example may be further improved upon via amplitude modulation and pulsing.

### INVENTIVE EXAMPLE 1B (Acoustic Percussion)

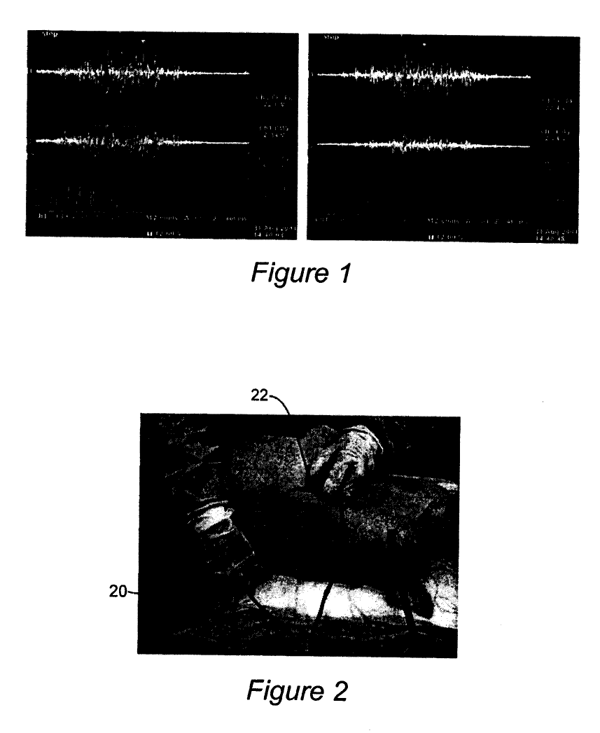

Percussion of the sternum while simultaneously ausculating the anterio chest on the side of the suspected pneumothorax results in an exaggerated chest resonance due to the excessive air. Chest resonance is about 8 Hz. By using pulsed AM of 8 Hz by 45 Hz an inexpensive shaker can be used to derive the effect. In this application a different transducer is used, according to the same basic principle of Example 1A. An example of the waveforms for acoustic percussion is in FIG. 1.

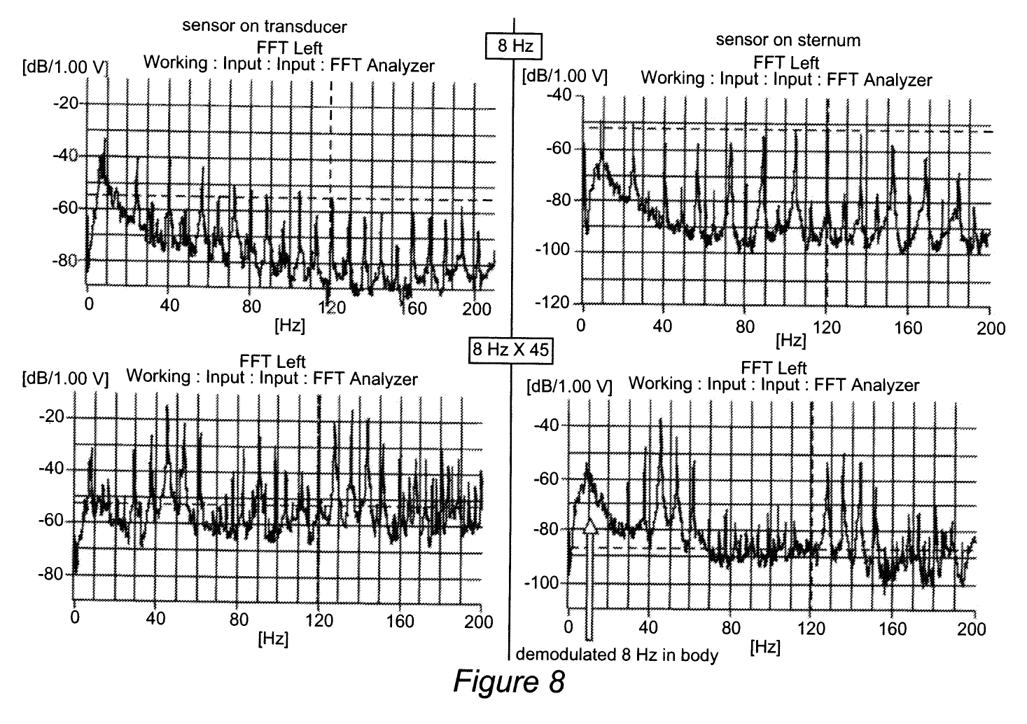

In the upper left panel of FIG. 8 is the spectrum of a 8 Hz tone burst as measured with an accelerometer on the transducer. Below is a carrier of 45 Hz modulated by 8 Hz. Note in the right panel, the 8 Hz is demodulated on my sternum at about the same level (intensity) as the 8 Hz tone unmodulated. Eight Hz will drive the chest into resonance so that the audible percussive effect for diagnosing pneumothorax can be achieved.

These Examples show that low frequency ultrasound as well as sonic energy can be used to diagnosis pneumothorax and hemothorax. This diagnostic use is a new use for low frequency ultrasound. The point of separation of the acoustic transmission about 30 kHz, a point where the wavelength and the tissue depth interact has been discovered by the present inventors and is being used for novel diagnostic methods and tissue analysis methods, which advantageously can be performed non-invasively.

Additionally, Example 1B shows that the inventors' percussion technique, induced acoustically, can be used to heighten the value of sonic and ultrasonic examination.

### COMPARATIVE EXAMPLE 2

Imaging ultrasound technology has become more popular in the trauma room primarily for the purpose of identifying blood in the chest cavity. In a retrospective analysis of 245 patients who received an ultrasound for assessment of the abdomen from a previous study, the average ultrasound examination lasted ˜4 minutes. Ultrasound can detect as little as 20 ml of fluid in the chesty cavity. X-rays cannot detect hemothorax unless at least 175 mL of fluid is present in the chest cavity. While ultrasound machines are reliable in determining hemothorax, they are bulky and expensive, which limits their use to only offices or hospitals. Imaging ultrasound uses frequencies greater than 1 MHz, thus requiring large-scale equipment.

### EXAMPLE 2

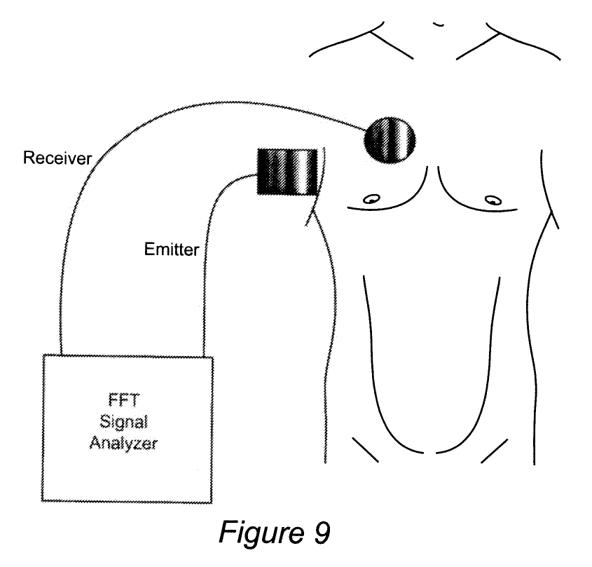

In this inventive example, a fast method and device capable of diagnosing pneumothorax uses high-frequency white noise sent through the chest cavity. The innovative portable pneumothorax detecting device of this example uses a transducer that emits a 10 Hz-50 kHz continuous band white noise sound. (FIG. 9) The pneumothorax detecting device generates vibro-acoustic wave frequencies, then transmits them from an emitter transducer to a receiver transducer located on the opposite side of the thorax. The receiving transducer, placed on the opposite side of the chest, senses the signal and sends the signal back to the real-time spectral analyzer for analysis. The spectral analyzer then computer the Fast Fourier Transform (FFT) on the signal and the result is displayed. The result indicates if pneumothorax or hemothorax is present. The velocity of the signal changes, due to attenuation, through different tissues inside the chest. When either air or fluid is present in the chest cavity, these change the acoustical characteristics of the chest. Therefore, in individuals with pneumothorax, the white noise should travel slower than in a healthy individual. If hemothorax is present, the sound waves will travel faster because of the fluid present in the pleural space.

In this experimentation, a Hewlett Packard 3561 A real-time spectral analyzer was used to produce and analyze the signal. The HP 3561A is a dynamic signal analyzer that performs fast Fourier transformation (FFT). The FFT output allows the user to view the frequency response of the calculated data. During this experimentation, the HP 3561A was used to produce adjustable frequencies to be sent to the source transducer. Different frequencies and amplitudes of sound waves were used, however, the use of a 550 Watt amplifier produced repeatable results. The amplifier was coupled to the noise source of the real-time signal analyzer to increase the signal strength. The HP3561A was adjusted as follows:

Signal processing =Narrow Band (401 lines)

RMS=30

Frequency width=50,000 Hz

Source=White Noise

Source intensity=0 dB

Window=Single

Wave=Sinusoidal

The resulting signal emitted by the HP3561A was a 10 Hz-50 kHz continuous wide band white noise signal. The flat frequency source allows for constant frequency resolution. During the experimentation, the resolution was equal to 50,000/401=125 Hz/line. Each line was equivalent to one discrete data point. This was an adequate resolution because the frequency range was so great. Although the signal analyzer is capable of analyzing 100 kHz signals, we found frequencies greater than 50 kHz did not propagate through the body effectively. The full-screen display mode allowed the information to be seen in more detail on the screen. Digital averaging was used to improve the precision of the signal-to-noise ratio of the measurements. The peak value can be held at every position. Root mean square (RMS) averaging was used to improve the statistical precision of the signals that constantly vary in amplitudes. RMS averaging was used to reduce the amplitude of signals not related to the source (bodily physiological noise), but did not eliminate them. The harmonics of the signal are read 80 dB below a full scale fundamental.

The appropriate sound parameter was selected to ensure propagation through the body to the receiving transducer. Both the source and receiving transducer resemble standard speakers and microphones. The head of the transducer is similar to the diaphragm of a microphone. When sound waves hit, the diaphragm vibrates back and forth. The diaphragm is attached to a coil that vibrates along with the diaphragm. Surrounded by the coil is a stationary magnet. As the coil vibrates back and forth over the magnet, the flux through the coil changes, producing an alternating current electromagnetic field across the coil. This signal is sent to an amplifier and then sent out to speakers. Thus the sounds waves are converted into electricity. The signal analyzer is capable of sensing the voltage changes and displays the voltages as a graph in the frequency domain for the user.

Piezo-rod transducers produced by Materials Systems Inc. (MSI) (customized as requested to be held in the hand and operational over the frequency range of interest, with water-proof cabling and connector for use in this Example), were used to provide the data results in this experimentation. The transducers are cylindrical, waterproof and durable, and worked very well in emitting signals between 10 Hz-50 KHz. This range was tested through direct coupling of the emitter to the receiver. An accelerometer and FFT signal analyzer verified these results. The transducer is made of piezoelectric ceramic rods setup in a polymer matrix. (FIG. 30) Because the transducers were designed as hydrophones, making them completely waterproof, and because their impedance is matched to skin, the transducers are the ideal emitter and receiver for a diagnostic device for lung injury applications. For optimal coupling to the skin surface, the transducer's impedance should match that of the skin surface.

### EXAMPLE 2A (Balloon Testing)

The pneumothorax detecting device of Example 2 was initially tested on rubber party balloons filled first with air, then water. The emitting and receiving transducers were placed on either side of the six inch diameter balloon. This test was performed t simulate the similar chest acoustical changes when a pneumothorax is present. Then the balloon was filled with water and the test procedure was repeated, to simulate the acoustical attenuation that would occur when a hemothorax is present. Ideally, the signal would be more damped by the air balloon and less by the water balloon.

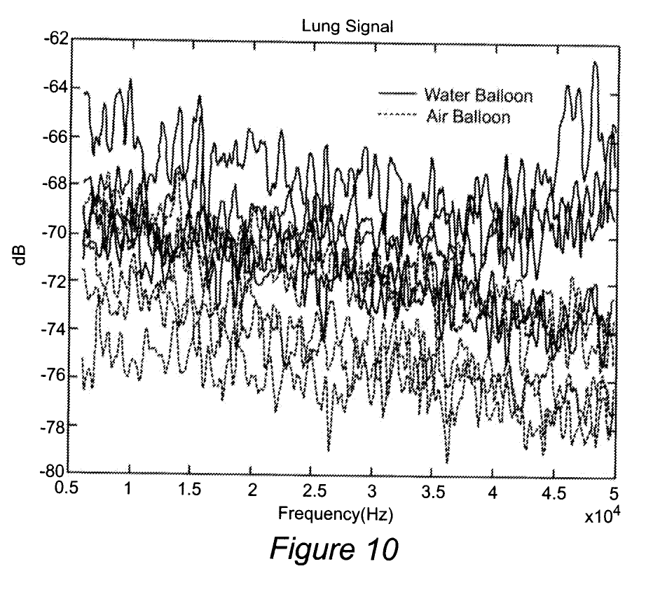

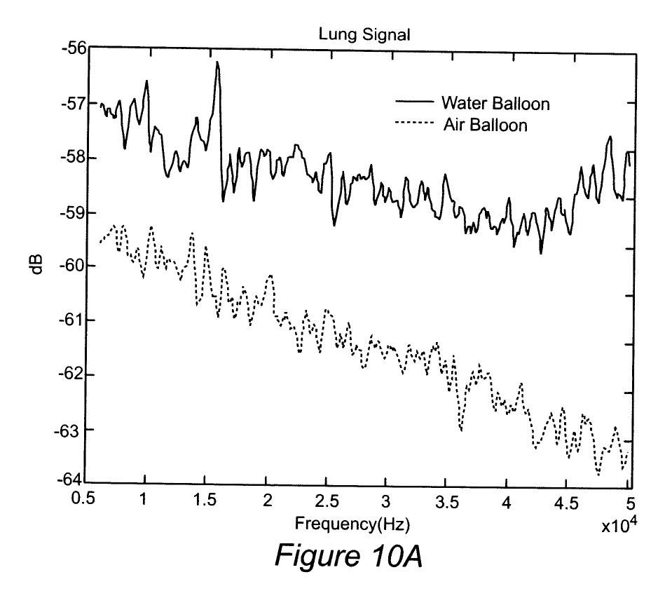

Data for testing the white noise acoustic waves on balloons is shown in FIG. 10. The difference in signals is more apparent through signal averaging. (FIG. 10A) The sound attenuation was much greater using the air-filled balloon than the water-filled balloon. The signal was slowed because when the signal encountered air, reflection, scattering, absorption, diffraction and refraction occurred. While the signal was impeded by both air and water, it was slowed much more by air.

### EXAMPLE 2B (Pig Testing)

Further testing of the pneumothorax detecting device of Example 2 was performed on adult pigs weighing ˜50 kgs. The pig's chest cavity is very similar to a human's in terms of volume and dimensions. However, the geometrical difference is that the pig's chest is more ‘V’ shaped than a human's (geometry and acoustic characteristics are more important than shape). This difference is seen chiefly in the sternum extending onwards. The larger sternum will increase the sound attenuation through the chest. However, this slight difference between pig and human thoraxes is of little significance in diagnosing pneumothorax or hemothorax with the device of Example 2, as long as the transducers are not placed directly on the sternum.

The source of the white noise frequency vibro-acoustic waves is positioned at a first location on the chest. Then the receiving transducer is positioned at a second location on the chest. These positioning on the chest were non-invasive. The sending transducer transmitted 50 kHz white noise frequency vibro-acoustic waves into the chest cavity. The waves were detected by the receiving transducer placed on the chest. The generated signal representative of the frequency response from the body was modified by the presence of either excess air or fluid in the pleural space. The system analyzed the frequency response signal by calculating a transfer function of the frequency response signal and examining the transfer function. The calculation took into account the spectrum of the source of high frequency vibro-acoustic waves and detected high frequency vibro-acoustic waves.

The system of Examples 2, 2B detects the presence of peaks in the response signal, indicative of resonance waves and detect the presence of dips in the response signal, indicative of anti-resonance waves. The system compares predetermined averaged values and decides whether or not pneumothorax or hemothorax may be present in the pleural space. By moving the receiving and source transducer to different points on the chest and getting multiple readings, the user can get a more accurate prediction. Moving the detector to a third location can enhance the resonance portion of the response signal and diminish the anti-resonance portion of the response signal. The recording method was performed through steps as follows: 1—Position a source of high frequency vibro-acoustic waves at a first location on the chest; 2—Position a detecting transducer at a second location of the body; 3—Transmit a high frequency vibro-acoustic wave(s) into the first location of the body; 4—Detect the transmitted high frequency vibro-acoustic waves at the second location on the body; 5—Generate a signal representative of the frequency response of the body resulting from the presence of air or fluid present in the pleural space; 6—Analyze the frequency response by calculating the Fast Fourier Transform of the attenuated signal; 7—Using the signal averages, determine whether the chest cavity has pneumothorax, hemothorax, or is healthy.

### Results

In all of the collected data for examples 2-2B, the intensity of the signal attenuation was measured by the acceleration of the signal (1 m/s²). The attenuation was measured in dB (re: 1V rms). The acceleration is the speed that the waves transmit through the chest cavity. The data obtained showed that acoustic impedance and velocity determine chest acoustical characteristics. The white noise signal travels through the chest faster when hemothorax is present and less when pneumothorax is present.

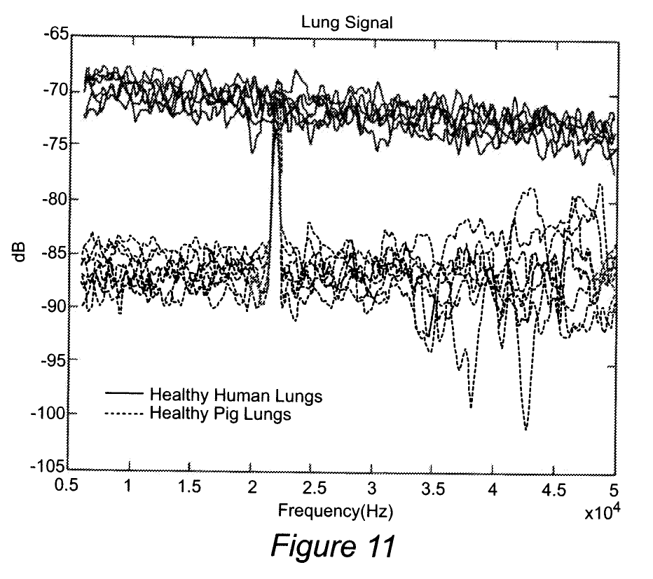

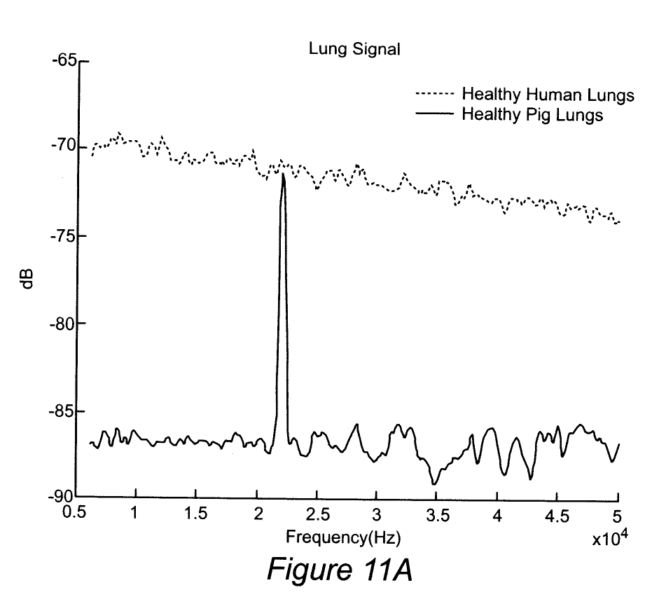

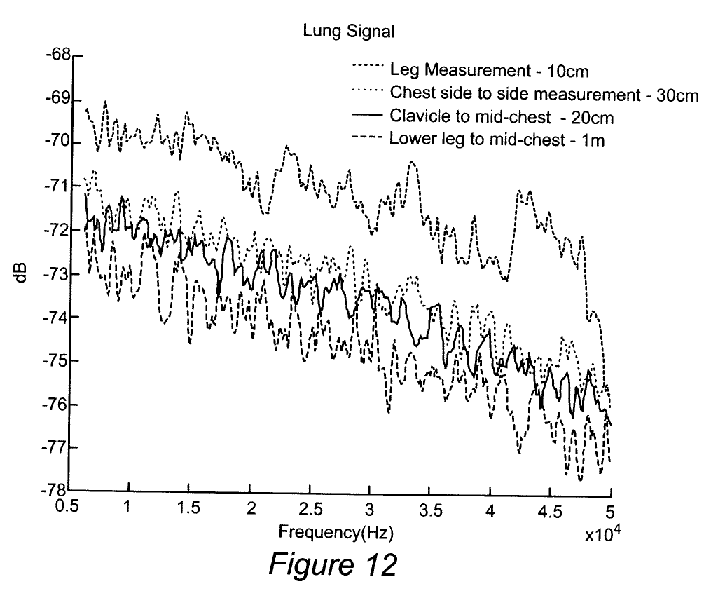

So that the results of experimentation on pigs in Example 2B may be better appreciated, comparison between healthy human chest attenuation versus healthy pig chest is shown in FIG. 11. The difference in signals is more apparent through signal averaging. (FIG. 11A) Signal attenuation comparisons are shown in FIG. 12; this data relates the vibro-acoustic impedance to dB/cm. Because only lower threshold ultrasound (50 kHz) was used, and because of the heterogeneity of the chest, attenuation difference was relatively small between each tissue. The computed average of the results are: 10 cm diameter leg=−71.16, 30 cm chest sides=−73.15, 20 cm clavicle to mid-chest=−73.60, and I m lower leg to mid-chest=−74.63. Thus, at 50 kHz there is not a large difference in the impedance of each of the tissues. In the tests which compared the side-to-side measurements of the chest (30 cm), the transducers were aimed directly at each other. In the tests which compared the clavicle-to-mid-chest measurements (20 cm), the transducers were held at right angles. The angled transducer, along with the clavicle bone impedance, account for why there is less measured attenuation over 30 cm than over the 20 cm experiment.

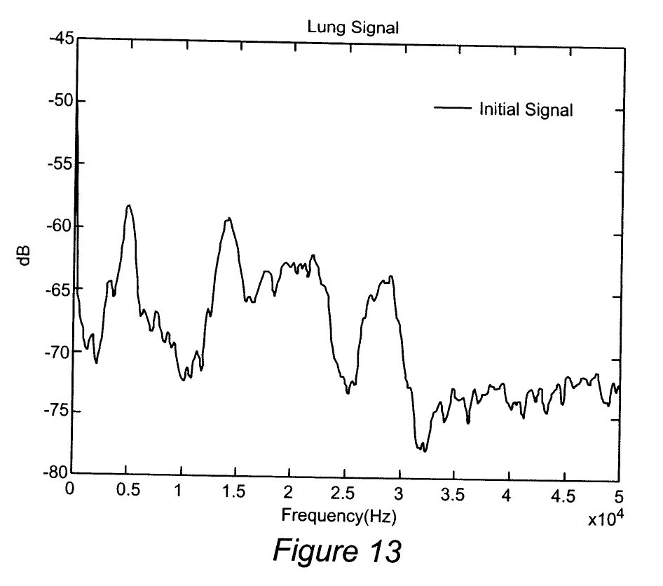

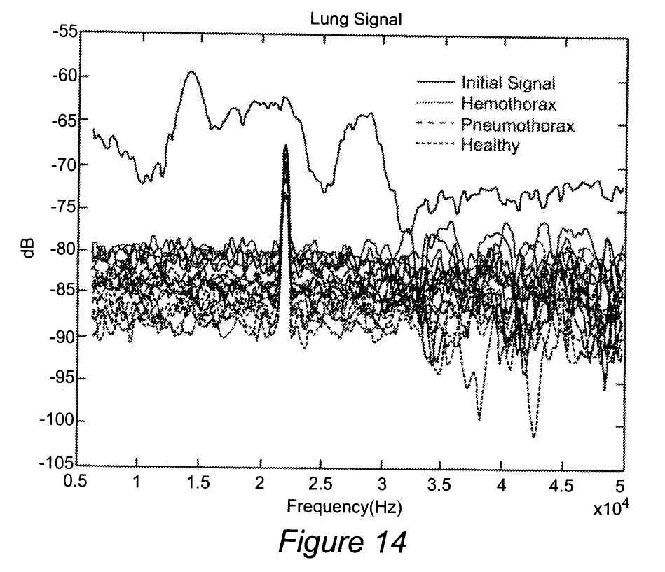

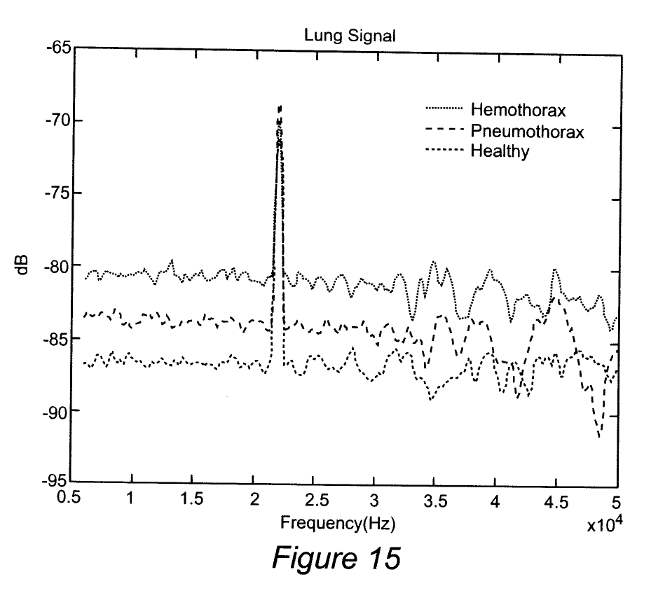

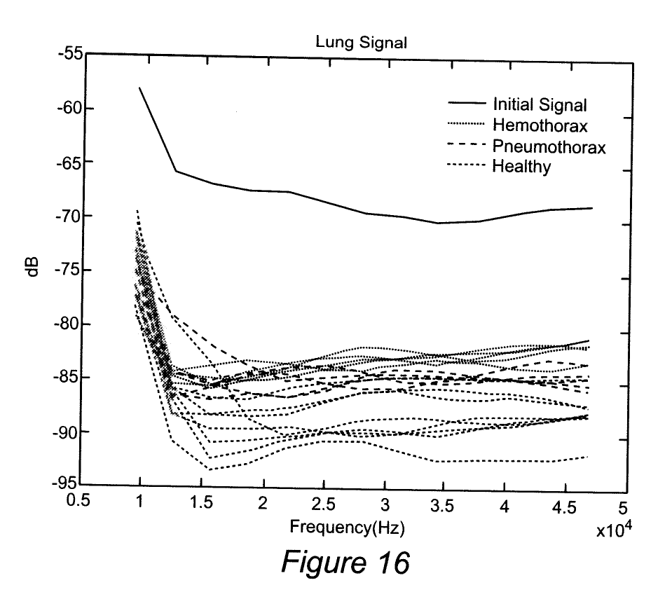

The initial signal produced by the signal analyzer was a white noise acoustic wave between 10 Hz-50 kHz (FIG. 13). Multiple different readings were made using different transducer arrangements and positions. All the recordings using the high range frequency transducer are shown in FIG. 14. Using these data, the average values were: initial signal=−69.4073 dB, healthy=−86.5234 dB, pneumothorax=−84.2403 dB, and hemothorax=−81.0091 dB. This data evidently proves the different ranges in attenuation for each of the chest cavity disease states. The differences between each pleural space situation are evident in FIG. 15. Lower frequencies also proved the tests. (FIG. 16.)

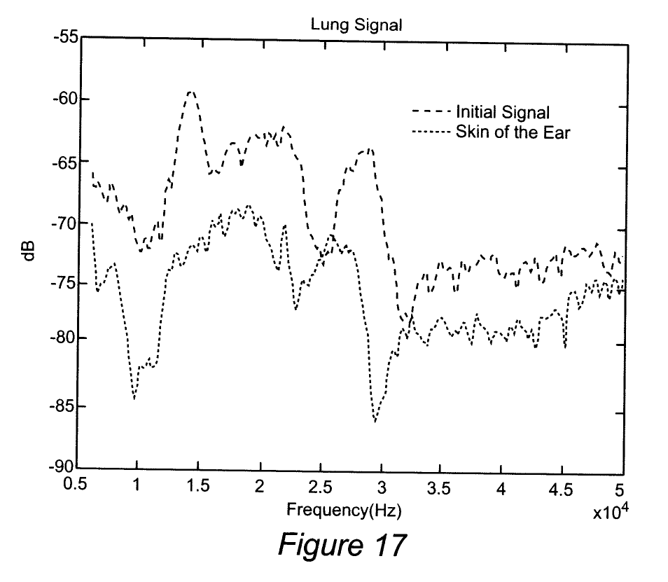

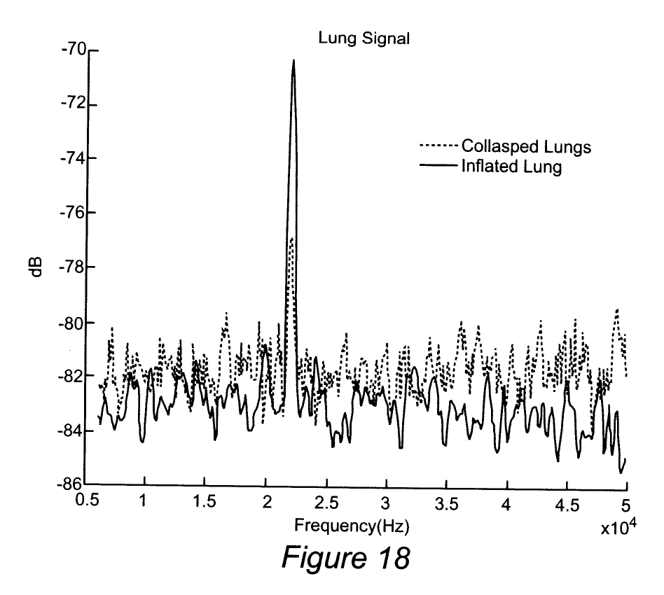

The impedance of skin was tested by connecting the transducers to the pig's ear. The results showed that all the frequencies passed through the skin. (FIG. 17.) The pig's lungs were removed so the transducers could measure their impedance directly. The relationship between the inflated and collapsed lung is shown in FIG. 18. The collapsed lung average value is −81.64 dB and the inflated lung average value is −82.88 dB. This data shows that the acoustic signal travels slower when a pocket of air is present.

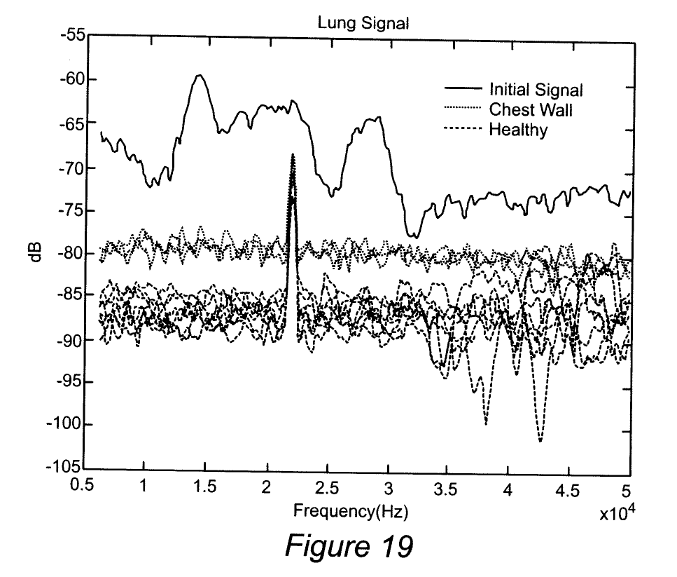

The chest wall itself was tested for its impedance (FIG. 19), by placing one transducer on the skin on the center of the chest and the other inside the pig's chest on the pleural layer. This determined the damping of the skin, ribs, intercostal muscles, chest muscles, and other tissue between the skin and the beginning of the pleural space. The average sound intensity for the chest wall was −79.63 dB. Thus, only minimal damping occurred from the chest wall itself. The results for all tests produced different attenuation values. The results clearly show the different levels in attenuation of a signal as it travels through different media, such as air, water, skin, lung tissue, the chest wall, and the chest cavity itself.

These examples show that by emitting vibro-acoustic waves through the chest cavity, followed by analysis of the propagated attenuation of the signal, presence of penumothorax or hemothorax can be diagnosed. By using low frequency vibratory ultrasound in the range from 10 Hz to 50 kHz, a portable neumothorax device is provided. Less power is needed to produce the lower amplified frequencies, thus making the device less dependent on a large voltage source, and hence providing more portability.

### Discussion

Sound is a wave phenomenon. Sound speed is a product of a wave frequency f and wavelength l and related by the equation a=f*l. Unlike electromagnetic waves, such as light waves, sound waves cannot travel through a vacuum, and must travel through a medium. Sound waves are longitudinal waves which alternate compressing and expanding the medium through which they are traveling. The wavelength of sound waves is the distance between successive expansions or compressions. The speed of sound depends on the compressibility and expandability of the medium through which it is traveling. The half value layer property of materials, related to wavelength, describes the distance sound waves travel before their intensity gets reduced by half.

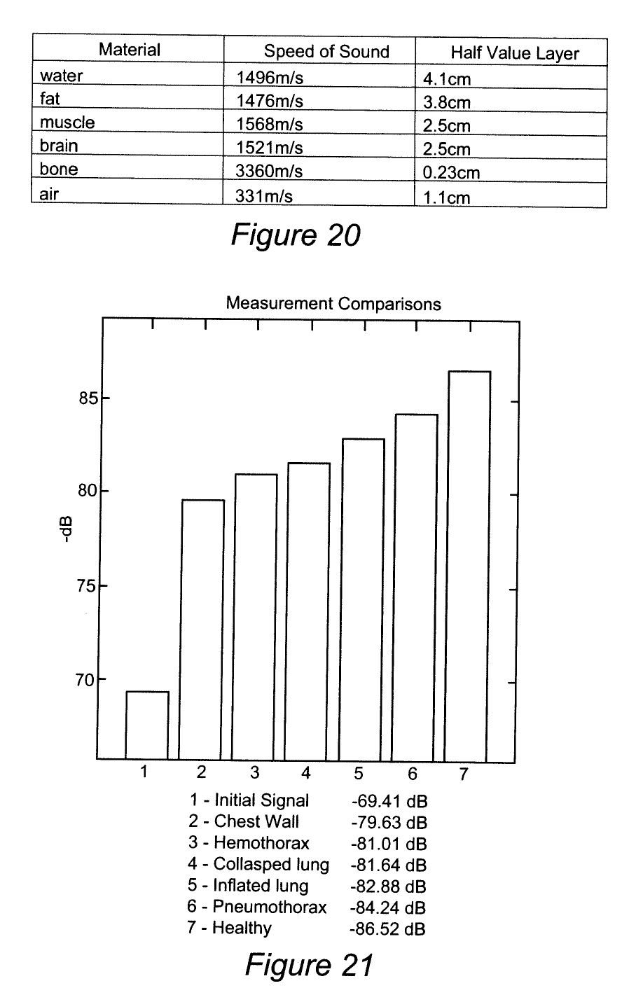

Sound travels through different mediums at varying speeds and half value layers. (FIG. 20) These different speeds justify the separation of attenuation values shown in the results of this example. Furthermore, the characteristic impedance Z₀=p*c. Thus, the density of a medium is related directly to its impedance. This explains how sound travels faster through water at 1496m/s and slower through air at 331 m/s. This further explains how the vibro-acoustic signal emitted by the transducer in this example is less impeded when it encounters fluid in the pleural space then when it encounters air.

The average attenuation due to impedance for all of the experiments in Examples 2-2B is shown in FIG. 21. The chest wall impedance is due to the intercostals muscles, the rib bones, and the pleural layer. The fluid present during the hemothorax tests significantly permits signal propagation, compared to the more impeded pneumothorax and healthy chest cavity tests. The greater attenuation produced in the inflated lung, compared to the collapsed lung, is due to the greater reflection and absorption of air.

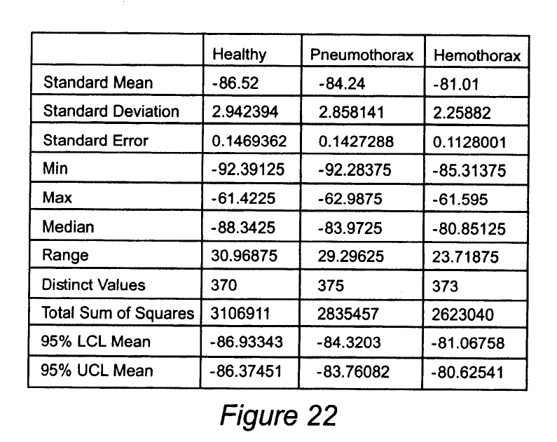

Statistical analysis was computed for the testing results from the pneumothorax, hemothorax, and healthy chest cavity raw data (FIG. 22). There is a discernible difference between the means for each test. The standard deviation from the mean is similar in each case, showing that the method to record the values was constant. Likewise, the standard error of the mean is similar for each instance. This is the estimated standard deviation from the distribution of sample means for an infinite population (Hintze). Thus, the mean was calculated with minimal error in each case, proving there is a difference in attenuation of the means.

The minimum and maximum values derive from the spike in data which occurs at 22 kHz (FIG. 14), and from the low frequency interference. The range, which is equal to max-min, is also produced by external interference of the experiment. The median value, which is the 50^{th} percentile value of the sorted data, shows the same degree of difference in each test as the sampled mean. This data, along with the difference in mean values and total sum of squares, proves that there is a shift in attenuation for each case. The similarity in distinct values further proves that the data was recorded in a similar method. Of the 401 discrete data values, ˜370 were unique values and not repeated. The 95% lower and upper confidence levels of the mean estimate the mean based on a t-distribution with n−1 degrees of freedom (Hintze). The statistical results (FIG. 22) show that the mean for each case lies within its own confidence level, and that no overlapping of confidence levels occurs. This ensures that the mean of the attenuation differences are greater than 95% accurate within its confidence levels.

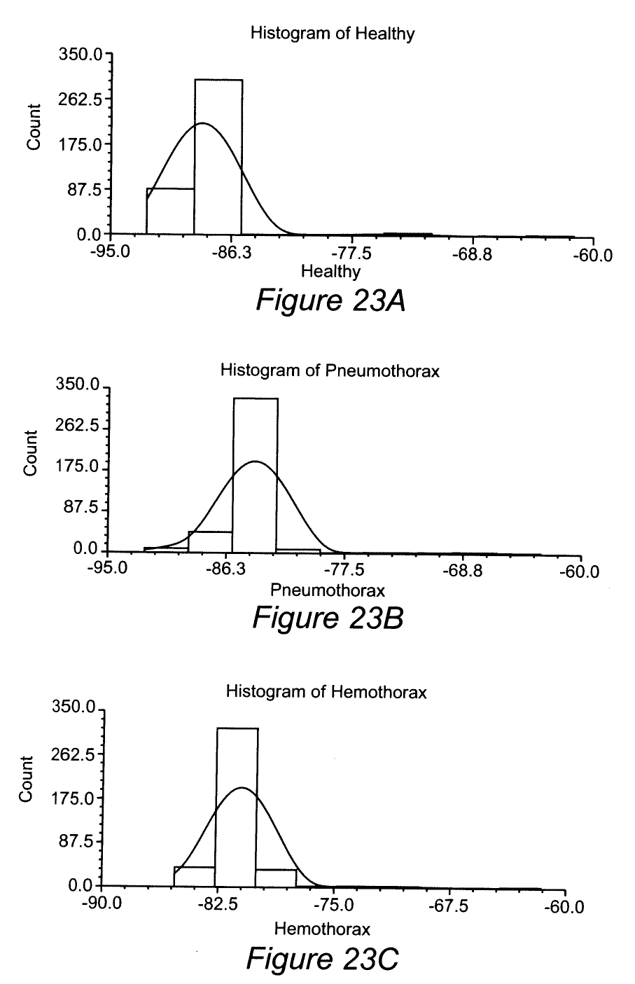

Histograms for each attenuation measurement average are shown in FIG. 23. The error from the spike encountered at 22 kHz produces the lengthening of the curve to the right. While the ideal shape is a symmetrical bell-shaped graph, the large box containing the majority is spaced out differently in each case. This proves the different attenuation data results have similar distribution frequency, although the means vary.

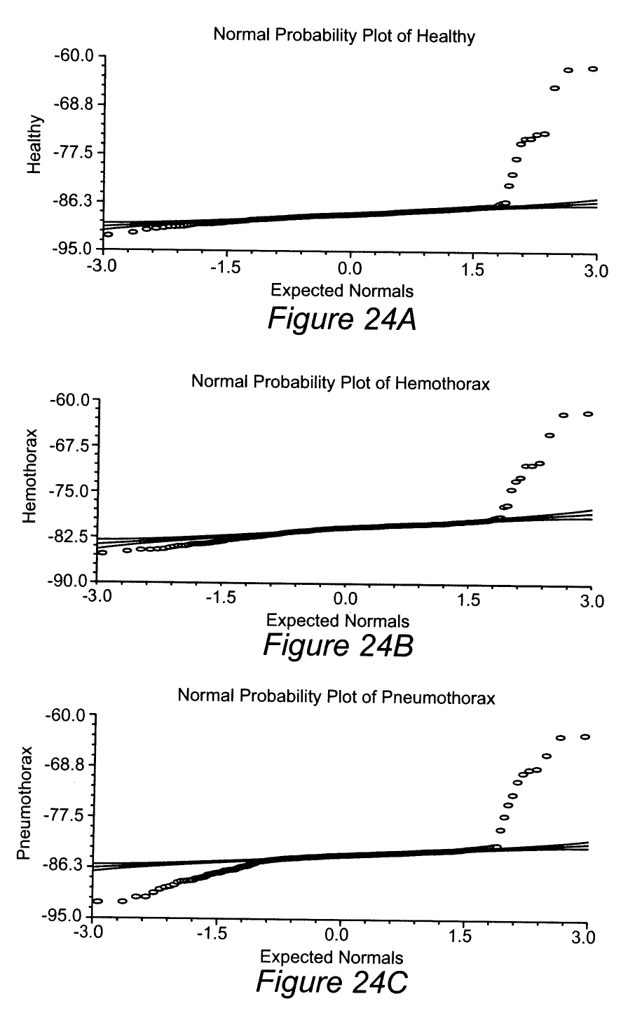

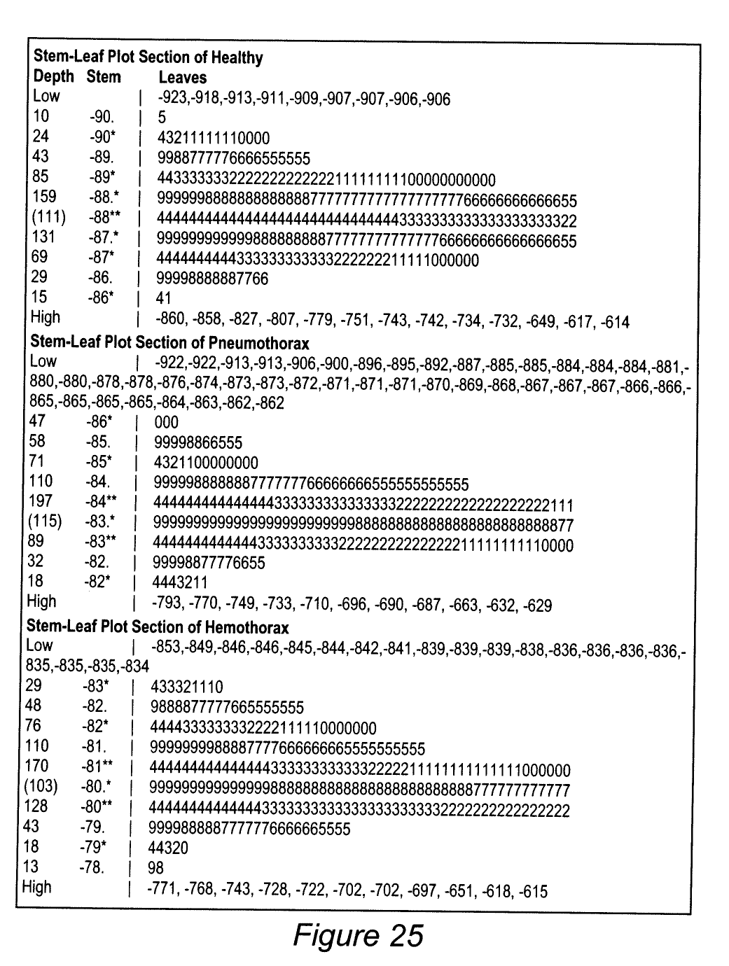

The probability plot of the data is shown in FIG. 24. The stragglers on either side of the plots show outliers in the data from the external frequency interference. The similar slopes in lines display the similar normality of each data set. Further comparison of the data is portrayed in the Stem Leaf chart (FIG. 25). The stems of each data set occur from −90 to −86 (healthy chest cavity), −86 to −82 (pneumothorax), and −83 to −78 (hemothorax). These differences confirm the separation in values, and the similarity in leaf structure for each test proves the similar distribution of data from the mean.

Using the statistical data from FIG. 22, the probability of incorrectly diagnosing pneumothorax or hemothorax from the computed data can be determined. The normal parameter Z is calculated by the equation Z=(χ−μ)/σ² where χ is the testing value, μ is the mean, and σ² is the variance. The probability of the means for attenuation overlapping is found by Z look-up tables. The probability that the device successfully diagnoses that the patient is healthy, and that he does not have hemothorax or penumothorax, results in a Z value of 0.774, or probability 0.781. The probability that the device successfully diagnoses penumothorax, and that the patient's chest cavity is not healthy and no hemothorax is present results in a Z value of 1.06, or a probability of 0.645. The probability that a hemothorax is present, and that the patient is not healthy and does not have a pneumothorax results in a Z value of 1.873, or a probability of 0.923. Because the difference between attenuations of the normal chest cavity hemothorax measurements was so great, the probability that the device correctly diagnoses that the patient has a hemothorax and does not have a healthy chest cavity is 0.969.

Therefore, the probability is higher that the device will detect hemothorax than pneumothorax. However, because physicians perform the same chest tube insertion to remove either air or fluid from the pleural space, the device can be calibrated to only detect a change in chest cavity attenuation measurements, regardless of specific air or fluid diagnosis. The calculated mean and variance measurements for the pneumothorax and hemothorax data can be averaged. The resulting probability that the device detects either a pneumothorax or a hemothorax, and that pneumothorax or hemothorax is present, is 0.907. Likewise the probability that the device incorrectly diagnoses that the patient is healthy, although a pneumothorax or hemothorax is present, is 1−0.907=0.0927. Thus, the device of Example 2B has a very high accuracy of 91%.

### EXAMPLE 2C (Increased Signal to Noise Ratio)

To further improve the accuracy of the device of Example 2B, the signal to noise ratio is increased. Signal to noise ratio determines the signal strength compared to the background noise. The equation to determine signal to noise ratio (SNR) is: −R=20(Log signal/noise)dB. The ratio R determines the relative useful information in relation to the useless noise carried in the signal. Thus, a signal with a higher R value will have a better quality signal. R is calculated in decibels (dB), which shows that noise greatly affects R. Therefore, if the signal intensity equals the noise intensity, then R is 0 and the signal will be essentially unreadable. The HP3561A signal analyzer effectively reduce noise with the square root of the number of averages. The signal may be strengthened further with greater amplification. However, the resulting increase in power consumption of the device and increased risk factor to the patient brings a trade-off.

### EXAMPLE 2D

In the experiments herein, the graphs were computed digitally by the HP 3561A signal analyzer which uses the fast Fourier transform algorithm to analyze the received signals. Any periodic waveform can be written as a sum of infinite sinusoidal terms. These infinite sinusoidal terms are Fourier series, and the process of finding them is Fourier analysis. Most approximations to original waveforms can be found with a limited number of sinusoidal terms. Computing Fourier analysis using integration would take a very long time to complete. Therefore the fast Fourier transform method is used. This computer algorithm uses look-up tables to quickly produce results in real time. Therefore, in the experiments herein, data could be quickly recorded after positioning the transducers.

In the experiments herein a frequency band of 10 Hz to 50 kHz was monitored. Two samples per time-dependent cycle at the maximum frequency is the lower theoretical limit for sampling, but the practical minimum rate is approximately 2.5 samples per cycle, which ensures that low-level frequency signals do not show up in the monitored frequency band. The frequency analyzer uses half as many frequency lines as data points because each line contains the real and imaginary data. The real values are the amplitude and the imaginary values are the phase.

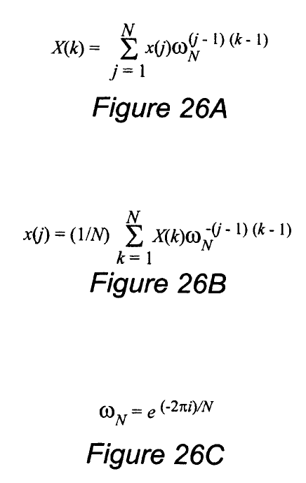

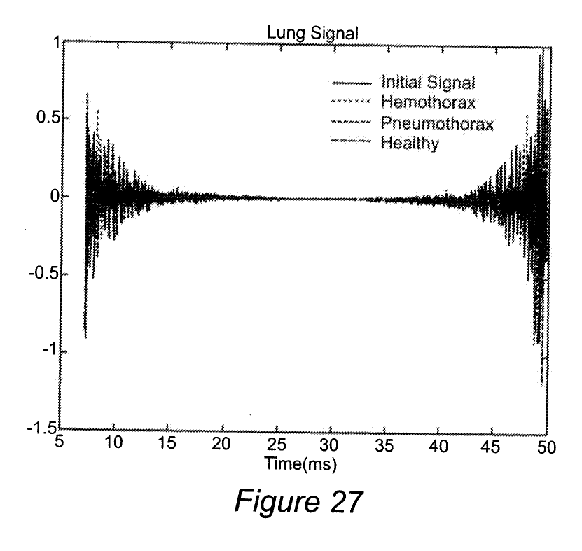

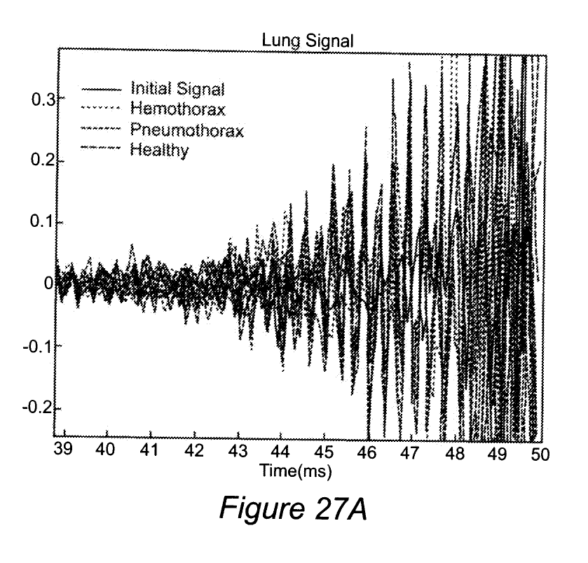

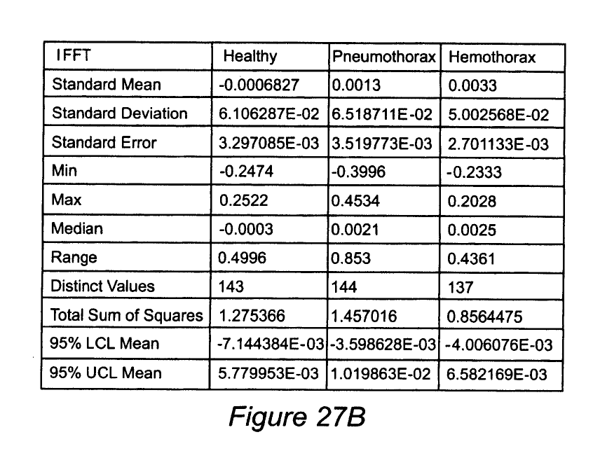

A further comparison test of the differences in measured attenuation of the data was performed using Fourier series analysis. MATLAB's inverse fast Fourier function is capable of switching from the frequency domain to the time domain. MATLAB uses the equations in FIG. 26 to compute the inverse fast Fourier transform algorithm. Equation ‘a’ (FIG. 26) computes the discrete Fourier transform. Equation ‘b’ (FIG. 26) computes the inverse discrete Fourier transform and ‘N’ is the length of the vector. FIG. 26c is definition for FIG. 26b. The results of the inverse fast Fourier transform are shown in FIGS. 27-27A. This data shows that the average values are: initial signal=0.0096, healthy=0.00068, hemothorax=0.0033, and pneumothorax=0.0013 dB. This data evidently shows the differences in each test exist in the time domain. The statistical analysis of the inverse Fourier data is shown in FIG. 27B. The computed standard deviation, standard error, and unique values for each data set are all comparable, justifying the accuracy of the mean and the overall distribution of the data.

Using low frequency ultrasound for the pneumothorax detecting device has many advantages. The transducers themselves are not highly directional, allowing the user (e.g., physician) to merely hold them against the skin of the chest to produce accurate readings. The physician or other user does not need to hold each transducer at an exact angle to one another.

Also, by using low frequency ultrasound, the drawback of using higher frequency ultrasound, that more interference occurs, can be avoided. Fluids, such as water or blood, will not greatly impede the vibro-acoustic signal as they would with higher frequency imaging ultrasound.

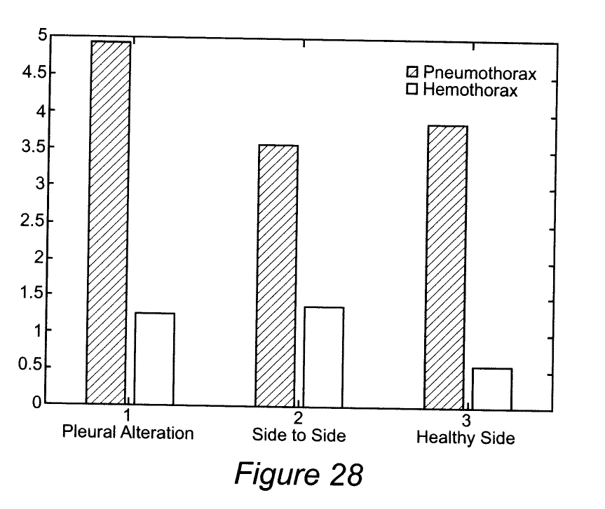

During the testing herein, pneumothorax and hemothorax were created on the left side of the pig, leaving the right pleural space intact. After each change of pleural space was produced measurements were taken with the transducers on both sides of the chest cavity, and from one side of the chest to the other. The results (FIG. 28) illustrate the increase in attenuation of the side in which the pleural space has been altered. Pneumothorax produces the greatest attenuation, because of the greater impedance of the air in the pleural space. The side-to-side measurements show an increase in attenuation for hemothorax, but a decrease in attenuation for pneumothorax. Therefore, side-to-side measurements are not practical measurement techniques. On the other hand, placing both transducers on one side of the chest produces valuable data for the pneumothorax detecting device to analyze.

### EXAMPLE 2E

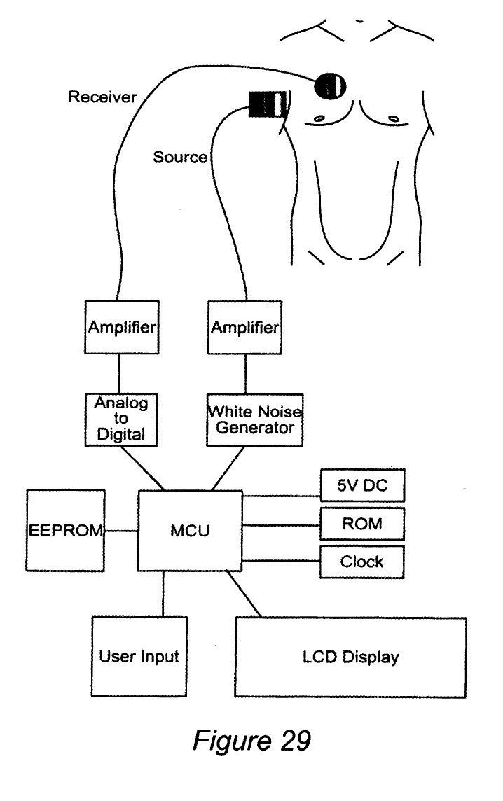

A device to measure pneumothorax is shown in FIG. 29. The microcontroller unit (MCU) is the main processor used. When the user presses the start button, the white noise generator produces a signal. This signal is increased by the amplifier and outputted by the source transducer. The signal travels through the chest cavity and is measured with the receiving transducer. This new signal is amplified by an amplifier and converted into binary representation through the analog-to-digital converter. This data is sent back into the MCU and saved into EEPROM memory (such as, e.g., a rewritable memory chip that retains data without needing a power source).

Preferably the memory is very power efficient, so that a 5V battery runs the device. The MCU instructions are contained in an external ROM. The ROM preferably is replaceable, making the device's program structure updatable. In a case where the frequency produced is 50 kHz, a clock frequency of at least twice that should be used. However, because modem MCU chips are small, low cost, and can be run at speeds of 4 MHz easily, measuring 50 kHz is simple. The device preferably has I/O buttons such as start, stop, reset. The saved memory may be displayed on a small LCD display screen and the MCU may output its prediction of the whether the pleural space contains air, contains fluid, or is healthy.

For constructing the device of FIG. 29, there may be used an FFT signal analyzer such as the portable FFT signal analyzer sold by Rion Co. of Japan, which can measure frequencies within the range 100 Hz-80 kHz. Rion's analyzer has a 192×128 dot LCD screen and a USB port for laptop data uploading. Thus, the screen is large enough to view possible attenuation differences seen when measuring the chest cavity.

Transducers produced by Material Systems Inc. work well in this application because of their improved impedance match to water, increased signal to noise ratio, increased sensitivity, and broader bandwidth, resulting in greater resolution.

Applied Instruments, Inc. produces a handheld signal generator to produce the white noise signal for the emitting transducer.

Portable 500 Watt amplifiers are made by many different manufacturers.

### EXAMPLE 2F

At frequencies below 10 kHz, the lungs act as a foam material because sound wavelengths are greater than alveolar size. The speed of sound is relatively slow, about 50 m/s. In air the speed of sound is much faster, 330 m/s. This is why 50 kHz range frequencies were selected to propagate through the chest cavity. When pneumothorax is present, free air in the pleural space will have relatively minimal acoustic damping and exhibit a resonant response to excitation. The size of the pneumothorax determines the resonant frequencies. When pneumothorax is present there will also be an increase in high frequency sounds because there is reduced damping beneath the chest wall. From experimental data, we have concluded that the frequency response of the thorax is changed due to the presence of enclosed air.

White noise was chosen as the noise source for the transducer because of its wide frequency range.

Alternately, pure tones and pulse signals can be used, at specific frequencies. Pulse signals offer less control over the level of vibratory energy input as a function of frequency. The ratio of sensor output to excitation input can be adjusted to minimize chest wall attenuations.

### EXAMPLE 2G (Transducer Placement)

Eight different placements of transducers were tested on a pig. From experimentation, we concluded that there was no ideal setup for placement of the transducers. While all the results in this experimental series were similar, placing the sending transducer on top of the chest and placing the receiving transducer on the side may produce less attenuation. Also, when the probes were held closer together, they generally produced less attenuation. Greater attenuation is preferred.

In this example, preferably the transducers are placed on the skin using echo transmission gel. Ideally the emitter transducer is placed on a rib or on the clavicle. The bones will be excited and the frequency will be conducted throughout the chest cavity. The receiving transducer preferably is placed on either another rib, opposite the source, or on another rib lower down from the source. This spacing assures that the propagated frequency signal will be measured after it is dampened by the tissues and possible pleural cavity changes.

The experiments in Examples 2-2G show that a low cost, non-invasive device that is able to produce quick results can be provided as a pneumothorax detecting device. Paramedics can use the inventive detecting device to quickly diagnose pneumothorax and military doctors and other personnel on battlefields could determine whether traumatic pneumothorax is present, so that a chest tube could be quickly inserted. Patients with COPD could use personal pneumothorax sensors to quickly conclude if a spontaneous pneumothorax has occurred. The data discussed herein show that using white noise propagated low level ultrasound frequency can correctly predict the air and fluid cavities present inside the chest.

### EXAMPLE 2H

A preferred device works by emitting vibro-acoustic waves via a transducer into the chest at a first location. The transducer introduces a standardized audible sound, gently, into the chest wall. A white noise generator producing vibro-acoustic waves in the range of 10 Hz to 50 kHz is connected to the transducer. A detecting acoustic sensor transducer is placed at a second location for detecting the transmitted attenuation of the signal. The detector identifies changes in the pleural space caused by the presence of air or fluid, and generates a signal representative of the frequency response. The attenuation of the signal occurs from its energy loss as it travels through different media. Therefore, the reflection and absorption that occurs due to air and fluid pockets in the pleural space is noticeably different compared to the healthy pleural space on the FFT signal analyzer. To predict more accurately the status of the pleural space, the emitting and receiving transducers preferably are positioned on either the left or right chest.

The pneumothorax and hemothorax detecting devices preferably are portable.

The detecting devices should be constructed for patient safety. The 50 kHz vibro-acoustic signals are not harmful to the patients. A safe power source should be used, such as a low-voltage DC power source which does not pose a shock hazard. Preferably transducers used are durable, waterproof, and reliable, in order to withstand rainwater at the scene of a traffic accident, or blood on a battleground.

### EXAMPLE 3

Referring to FIG. 31, a portable prototype non-invasive pneumothorax device is sized on the order of its longest dimension being ˜10 inches lengthwise (as shown), and comprising an accelerometer, transducer, pre-amplifier (“pre am”), battery (“batt”), noise generator (“noise”), Fast Fourier Transformer (“FFT”) and amplifier (“amp”). (Sine wave generator, amplitude modulator and pulser are not shown in FIG. 31.)

### EXAMPLE 4

This inventive example is according to the previous examples except that transducer placement for applying at least one wave or ultrasonic noise is on a patient's trachea. A measure is taken based on receiving signals on the patient's chest wall.

### EXAMPLE 5

In this inventive example, the inventive method of any previous example is used in conjunction with high frequency ultrasound for imaging purposes.

### EXAMPLE 6

The inventive method of any previous example is practiced in conjunction with high frequency ultrasound for imaging purposes.

While the invention has been described in terms of its preferred embodiments, those skilled in the art will recognize that the invention can be practiced with modification within the spirit and scope of the appended claims.

We claim:

1. A method for diagnosing pulmonary injury, comprising:
interrogating at least one pulmonary tissue which is within a patient with at least one wave having a frequency in a range of about 20 kHz to 50 kHz, wherein the at least one wave is selected from the group consisting of: a continuous wave, a pulsed wave, an amplitude modulated (AM) wave, and ultrasonic noise;
automatically detecting results of the interrogating step; from which detected results a diagnosis may be made of whether a pulmonary injury is present or absent in the patient.

2. The method of claim 1, wherein the wave has a frequency above 30 kHz.

3. The method of claim 1, wherein the interrogating comprises interrogating with a continuous wave.

4. The method of claim 1, wherein the interrogating comprises interrogating with a pulsed wave.

5. The method of claim 1, wherein the interrogating comprises interrogating with an AM wave.

6. The method of claim 1, wherein the interrogating comprises interrogating with an ultrasonic noise.

7. The method of claim 1, wherein the tissue interrogating is noninvasive.

8. The method of claim 1, including placing at least one probe on a region of soft tissue of a patient.

9. The method of claim 1, wherein the pulmonary injury is selected from the group consisting of pneumothorax, hemothorax, hemopneumothorax, pulmonary effusion and pulmonary contusion.

10. The method of claim 1, including diagnosing pneumothorax.

11. The method of claim 1, including diagnosing hemothorax or hemopneumothorax.

12. The method of claim 1, including a percussion step.

13. The method of claim 1, wherein the method is practiced using a handheld device and pneumothorax, hemothorax or hemopneumothorax is diagnosed.

14. The method of claim 1, wherein pneumothorax, hemothorax or hemopneumothorax may be diagnosed in an environment with high ambient airborne noise.

15. The method of claim 1, including a step of diagnosing whether pulmonary injury is present in the patient.

16. The method of claim 1, including an FFT step and/or spectral analysis step.

17. A portable medical device for diagnosis of lung injury, comprising:
a wave and/or noise generator generating at least wave having a frequency in a range of about 20 kHz to 50 kHz, wherein the at least one wave is selected from the group consisting of: a continuous wave, a pulsed wave, an amplitude modulated (AM) wave, and ultrasonic noise;
an interface through which the low frequency wave is delivered to at least one lung, and
a detection component which detects a signal that results from applying the low frequency wave to the lung.

18. The portable medical device of claim 17, wherein components inconsistent with the device having a portable size are excluded.

19. The portable medical device of claim 17, wherein the device is handheld size.

20. The portable medical device of claim 17, wherein the device is waterproof.

21. The portable medical device of claim 17, wherein the interface is an interface via which the wave is delivered non-invasively to a patient.

22. The portable medical device of claim 17, wherein the interface is an interface via which the wave is delivered via a region selected from the group consisting of: a trachea of a patient and a soft tissue region of a patient.

23. The portable medical device of claim 17, wherein the interface is an interface via which the low frequency wave is delivered via a trachea of the patient and detected on the thoracoabdominal or is received at the trachea after having been delivered to the patient's thoaracoabdominal surface.

24. The portable medical device of claim 17, wherein the interface is an interface via which the low frequency wave is delivered or received from an endotracheal tube which is in the patient's trachea and respectively received or delivered from the patient's thoracoabdominal surface.

25. The portable medical device of claim 17, wherein the interface is an interface via which the low frequency wave is delivered or received from a nasogastric tube which is in the patient's esophagus and stomach and respectively received or delivered from the patient's thoracoabdominal surface.

26. The portable medical device of claim 17, wherein the interface is an interface via which the low frequency wave is delivered or received from a mouth (oropharynx) of a patient.

27. The portable medical device of claim 17, wherein the wave and/or noise generator comprises a simulator system simulating at least one continuous wave.

28. The portable medical device of claim 17, wherein the wave and/or noise generator comprises a simulator system simulating at least one pulsed wave.

29. The portable medical device of claim 17, wherein the wave and/or noise generator comprises a simulator system simulating at least one demodulated wave.

30. The portable medical device of claim 17, wherein the wave and noise generator comprises a simulator system simulating at least one amplitude modulated (AM) wave.

31. The portable medical device of claim 17, wherein the wave and/or noise generator comprises a non parametric propagator.

32. The portable medical device of claim 17, wherein the wave and/or noise generator comprises a simulator system simulating ultrasonic noise.

33. A portable medical device for diagnosis of lung injury, comprising:
a wave and/or noise generator generating at least two different frequencies simultaneously,
an interface through which the at least two different frequencies simultaneously are delivered to at least one lung, and
a detection component which detects a signal that results from applying the at least two frequencies to the lung;
wherein components inconsistent with the device having a portable size are excluded.

34. The portable medical device of claim 33, each frequency being in a range of about 20 kHz to 50 kHz.

35. The portable medical device of claim 33, wherein each different frequency has a frequency source selected from the group consisting of: a continuous wave, a pulsed wave, an amplitude modulated (AM) wave, and ultrasonic noise.

36. The portable medical device of claim 35, comprising frequency sources that are the same.

37. The portable medical device of claim 35, comprising a mixture of different frequency sources.

38. The portable medical device of claim 33, including a sensor array for receiving signals.

39. A method of diagnosing lung injury, comprising:
generating at least two different wave and/or noise frequencies simultaneously,
through an interface simultaneously delivering the at least two different frequencies to at least one lung, and
detecting a signal that results from the at least two frequencies being delivered to the lung.

40. The method of claim 39, wherein the generating, delivering and detecting steps are practiced using portable equipment.
